(19) United States
(12) Patent Application Publication
(10) Pub. No.: US 2018/0001581 A1
(43) Pub. Date: Jan. 4, 2018

(54) COMPOSITIONS, SYSTEMS AND METHODS FOR PATIENT SPECIFIC OPHTHALMIC DEVICE

(71) Applicants: Jayant K. PATEL, Santa Monica, CA (US); Kieren J. PATEL, Evanston, IL (US); NORTHWESTERN UNIVERSITY, Evanston, IL (US); OPTICENT HEALTH DEVICE, INC., Evanston, IL (US)

(72) Inventors: Jayant K. Patel, Santa Monica, CA (US); Cheng Sun, Wilmette, IL (US); Hao F. Zhang, Deerfield, IL (US); Rushi K. Talati, Chicago, IL (US); Kieren J. Patel, Evanston, IL (US)

(21) Appl. No.: 15/543,490

(22) PCT Filed: Jan. 14, 2016
(86) PCT No.: PCT/US2016/013438

§ 371 Date: Jul. 13, 2017

Related U.S. Application Data
(60) Provisional application No. 62/103,416, filed on Jan. 14, 2015.
(60) Provisional application No. 62/207,126, filed on Aug. 19, 2015.
(60) Provisional application No. 62/239,335, filed on Oct. 9, 2015.

(51) Int. Cl. B29D 11/00; B29C 64/129; G02C 7/04; B29D 11/02; B33Y 80/00

(52) U.S. Cl. CPC B29D 11/00086; G02C 7/04; B29D 11/023; B29D 11/00961; B29C 64/129; A61F 2240/002; B33Y 80/00

(57) ABSTRACT

Systems, methods, and devices to fabricate one or more device components are disclosed. An example method includes fabricating one or more subject specific device components generated from receiving one or more images of one or more features of the first eye of the subject; designing a three dimensional virtual geometric model of the ophthalmic device using the one or more images; generating a plurality of virtual cross-sections of the three-dimensional virtual geometric model, wherein the cross-sections are defined by a set of physical parameters derived from the three-dimensional model; and fabricating the one or more subject specific features using the plurality of virtual cross-sections of the three dimensional model to direct an additive manufacturing method.

### CROSS-REFERENCE

This application claims the benefit of priority to PCT Patent Application No. PCT/US2016/013438, filed Jan. 14, 2016, U.S. patent application Ser. No. 62/103416, filed Jan. 14, 2015, U.S. patent application Ser. No. 62/207126 filed Aug. 19, 2015, and U.S. patent application Ser. No. 62/239335, filed Oct. 9, 2015, each of which is incorporated herein by reference in its entirety for all purposes.

### STATEMENT REGARDING FEDERALLY SPONSORED RESEARCH FOR DEVELOPMENT

This invention was made with government support under R01 EY019951 awarded by the National Institutes of Health; and CBET1055379 awarded by the National Science Foundation. The government has certain rights in the invention.

### BACKGROUND OF THE DISCLOSURE

Additive manufacturing, also known commonly as three-dimensional (3D) printing, has become a useful and important technique for manufacturing a variety of complex 3D structures. This method has been applied to the manufacturing of numerous devices and components, ranging from toys to jet engine parts. Generally, 3D printing provides an accessible and cost efficient method for generating customizable objects. Devices and components that were previously too difficult, or too costly to manufacture using traditional fabrication methods can now be prototyped or commercially produced using 3D printing technology.

Despite the potential of 3D printing (3DP), current 3D printed components have been limited by 3D printing resolution. Generally, 3D printing resolution reflects the ability to control the thickness of individual layers or material added to the object during the printing process. Limitations of the material and manufacturing time also commonly limit 3D printing resolution, thus limiting object resolution and manufacturing precision. While this limitation has not prevented the application of 3D printing to application such as toy manufacturing, it has prevented its application to fabrication of objects requiring high precision and resolution.

Ophthalmic devices, or ophthalmic medical devices for treatment or cosmetic use in the eye, are widespread and used for a variety of eye diseases and conditions. As of 2010, the World Health Organization (WHO) estimated that nearly 40 million people worldwide suffer from blindness; 246 million suffer from moderate to severe visual impairment; and 285 million live with some form of visual impairment. Nearly 51% of cases of visual impairment/blindness stem from cataracts, which require the use a type of ophthalmic device, an intraocular lens, for treatment. As a class, ophthalmic devices, ranging from intraocular lenses to ophthalmic stents, represent a common and important type of medical device critical in the treatment of a variety of visual impairment diseases.

Generally, ophthalmic devices require a higher degree of fabrication complexity and precision given the small and intricate structures of the eye. Additionally, many ophthalmic devices have an optical component and/or are involved in the correction of eyesight and therefore rely on precision manufacturing techniques to provide certain optical properties and quality. In some examples, ophthalmic devices are fabricated using a variety of molding and casting methods. However, these methods are limited and may not be easily amenable for cost efficient, time efficient, manufacturing of customized devices. The ability to customize ophthalmic devices may confer numerous benefits over non customized devices, including but not limited to, the ability for rapid prototyping of novel ophthalmic device designs, customization of patient specific devices for improved fit and device performance for disease or cosmetic treatment, manufacturing of devices from novel materials, and generation of novel engineered device designs, previously difficult or unachievable using existing fabrication methods. There is need in the art for customizable patient specific ophthalmic devices and methods for manufacturing these devices therein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of a device of this disclosure are set forth with particularity in the appended claims. A better understanding of the features and advantages of this disclosure will be obtained by reference to the following detailed description that sets forth illustrative examples, in which the principles of a device of this disclosure are utilized, and the accompanying drawings of which:

FIG. 1 is a flow diagram of an example method for fabrication of a 3D printed ophthalmic device.

FIG. 2 is a flow diagram of an example method for fabrication of a 3D printed ophthalmic device.

FIG. 3 is a schematic of a system for fabricating an ophthalmic device.

FIG. 4 illustrates an example 3D virtual geometric model of an ophthalmic device.

FIG. 5 is a diagram illustrating an example process from computer aided design to 3D printing to form 3D parts.

FIG. 6a is an image of Air Force target testing of an experimental 3D printed lens without post processing.

FIG. 6b is an image of Air Force target testing of an experimental 3D printed lens with polishing post processing.

FIG. 7a illustrates an example of wetting a 3D printed device with curable material.

FIG. 7b illustrates an example of dipping a 3D printed device in curable material.

FIG. 7c illustrates an example of curing or polymerizing curable material during a post processing reaction to smooth 3D printed layers.

FIG. 8a is an image of Air Force target testing of an example 3D printed lens post processed with resin curing.

FIG. 8b is an image of a printed lens on top of the Air Force target is on the right side of the image.

FIG. 9 is an image of an example lens modeled in a CAD software program.

FIG. 10 is flow diagram of an example process to fabricate a lens using a 3D printed mold.

FIG. 11 is a schematic of an OCT setup to image an eye or a lens.

FIG. 12a is an image of an example 3D printed accommodating intraocular lens design in CAD software.

FIG. 12b is a magnified image of a functional element of the example 3D printed accommodating intraocular lens design.

FIG. 12c is an image of 3D printed device, printed from the example design shown in FIG. 12a and FIG. 12b.

FIG. 13a is an image of an example 3D printed accommodating intraocular lens design in CAD software.

FIG. 13b is an image of an example 3D printed accommodating intraocular lens design in CAD software.

FIGS. 14a-b are images of an example 3D printed accommodating intraocular lens design in CAD software and side view of the design.

FIG. 15a is an image of an example 3D printed accommodating intraocular lens design in CAD software and various force measurements to determine displacement of the internal ring.

FIG. 15b is an image of an example 3D printed accommodating intraocular lens design in CAD software and various force measurements to determine displacement of the internal ring with a lens element.

FIG. 16 is an image of an example 3D printed accommodating intraocular lens design in CAD software and various force measurements to determine displacement of the internal ring.

FIG. 17 is a block diagram illustrating a first example architecture of a computer system that can be used in connection with systems, methods, and devices of this disclosure.

FIG. 18 is a diagram showing a network with a plurality of computer systems, and a plurality of cell phones and personal data assistants configured with systems, methods, and devices of this disclosure.

FIG. 19 is a diagram showing a network with a plurality of computer systems, and a plurality of cell phones and personal data assistants configured with systems, methods, and devices of this disclosure.

FIG. 20 is a diagram illustrating a first example a computer system that can be used in connection with one or more systems, methods, and devices of this disclosure, including handheld or mobile devices.

FIG. 21 is an image of an example 3D printed accommodating intraocular lens design in CAD software and side view of the design.

FIG. 22a is an image of example 3D patterns of a device printed using stereolithography.

FIG. 22b is an image of example 3D patterns of a device printed using stereolithography.

FIG. 22c is an image of example 3D layers printed using stereolithography.

FIG. 22d is an image of example 3D layers of a device printed using stereolithography.

FIG. 23a is an image of an example 3D printed intraocular lens without accompanying haptics.

FIG. 23b is an image of an example 3D printed intraocular lens haptics without an accompanying lens.

FIG. 23c is an image of an example 3D printed intraocular lens haptics and lens portion.

FIG. 24 is a schematic of 3D printing of an example ophthalmic contact lens device using a mold.

The following detailed description of certain examples of the present disclosure will be better understood when read in conjunction with the appended drawings. For the purpose of illustrating the disclosure, certain examples are shown in the drawings. It should be understood, however, that the present disclosure is not limited to the arrangements and instrumentality shown in the attached drawings.

### DETAILED DESCRIPTION OF THE DISCLOSURE

### I. General Overview

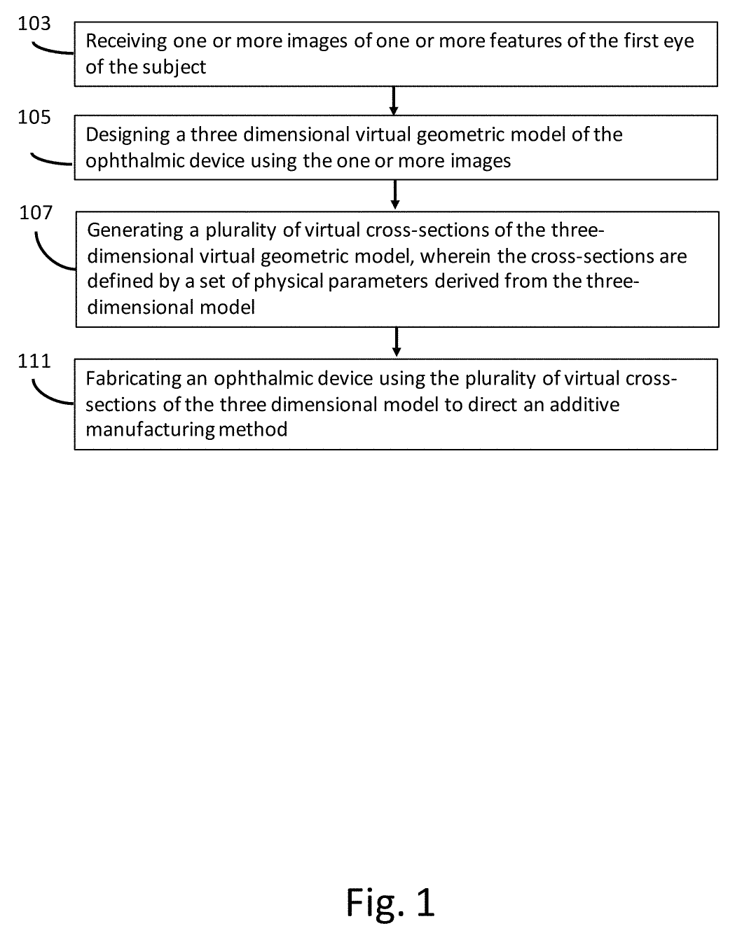

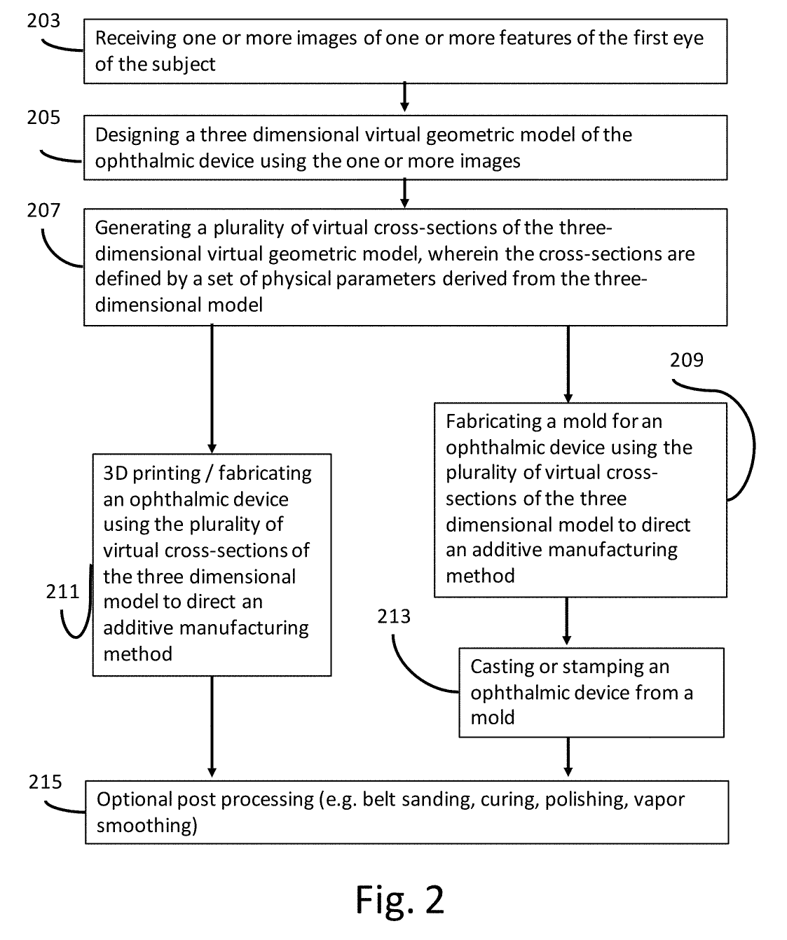

The devices, methods, and systems of the present disclosure provide a method, such as the methods illustrated in FIG. 1 and FIG. 2, for fabricating an ophthalmic device for a first eye of a subject, the method comprising receiving one or more images of one or more features of the first eye of the subject, 103, 203 designing a three dimensional virtual geometric model of the ophthalmic device using the one or more images, 105, 205, generating a plurality of virtual cross-sections of the three-dimensional virtual geometric model, wherein the cross-sections are defined by a set of physical parameters derived from the three-dimensional model, 107, 207 and fabricating the ophthalmic device using the plurality of virtual cross-sections of the three dimensional model to direct an additive manufacturing method, 111, 211. In some examples, such as in FIG. 2, after generating a plurality of virtual cross-sections of the three-dimensional virtual geometric model, wherein the cross-sections are defined by a set of physical parameters derived from the three-dimensional model, fabricating a mold for an ophthalmic device using the plurality of virtual cross-sections of the three dimensional model to direct an additive manufacturing method may be employed, 209, followed by casting or stamping an ophthalmic device from a mold, 213. An optional step of post processing, 215, (e.g. belt sanding, curing, polishing, vapor smoothing) may also be employed after fabrication.

The devices, methods, and systems of the present disclosure also provide for any suitable ophthalmic device wherein the device comprises one or more subject specific features device components generated fabricated from receiving one or more images of one or more features of the first eye of the subject; designing a three dimensional virtual geometric model of the ophthalmic device using the one or more images; generating a plurality of virtual cross-sections of the three-dimensional virtual geometric model, wherein the cross-sections are defined by a set of physical parameters derived from the three-dimensional model; and fabricating the one or more subject specific features device components using the plurality of virtual cross-sections of the three dimensional model to direct an additive manufacturing method.

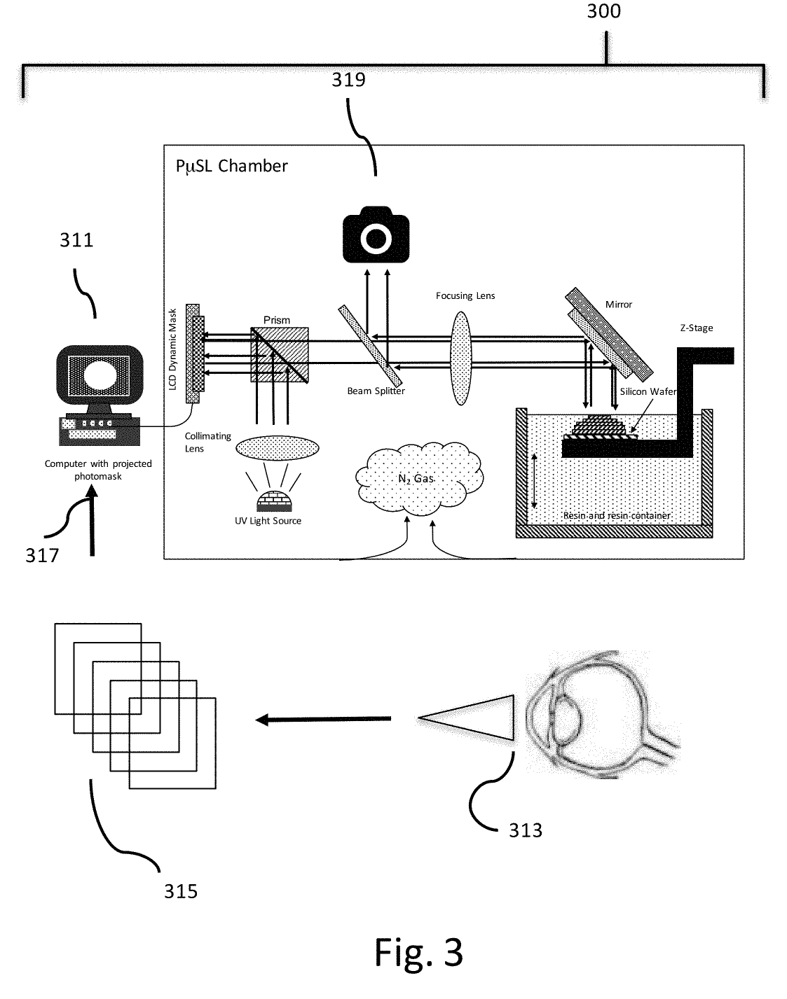

Additionally, the devices, methods, and systems of the present disclosure also provide, as shown in the example of FIG. 3, a system 300 including a computer system 311 configured to receive 317 one or more images 315 of features of the first eye of a subject 313. The computer system 311 is particularly programmed to process the received 317 image(s) 315 of the eye to form a three-dimensional geometric model of an ophthalmic device based on the image(s) 315. The particularly programmed computer system 311 further processes the model to mathematically slice the three-dimensional mathematical model into a plurality of cross-sections. Each cross section is defined by a set of physical parameters derived from the three-dimensional model. The computer system 311 provides the plurality of virtual cross-sections from the three-dimensional model to direct an additive manufacturing process such as using a Projection Micro-stereolithography system (PμSL) chamber 319. The chamber 319 receives the plurality of cross-sections of the model from the computer 311 and fabricates an ophthalmic device using the plurality of virtual cross-sections of the three-dimensional device model.

For example, the chamber 319 shown in FIG. 3 includes a dynamic mask 321 (e.g., a bitmap mask) generated based on the cross-sections from the computer 311. UV light 327 is directed through a collimating lens 325 and a prism 323. The light then passes through a beam splitter 320, a projection lens 341, and is reflected on a mirror 343 to a wafer 353, such as a silicon wafer, in a z-stage 345 basin or other receptacle with a supply of UV curable resin 347 to form an ophthalmic device. A camera 349 can be used to visualize an image on the wafer 353 in the chamber 319, which is filled with a gas 351, such as nitrogen gas.

Further examples of the system and methods used for fabricating an ophthalmic device using an additive manufacturing process are described herein.

### II. Types of Ophthalmic Devices

The devices, methods and systems of the present disclosure generally provide for any patient specific ophthalmic device that can be manufactured using one or more 3D printing technologies. In some examples, an ophthalmic device, or ophthalmic device component(s) may refer to any^{,} ophthalmic device that is capable of residing in or on the eye. Ophthalmic device and ophthalmic device component(s) maybe used interchangeably herein. In some examples, an ophthalmic device, or ophthalmic device component(s) may be a device that provides a type of treatment, monitoring, or diagnostics for one or more ophthalmic diseases, conditions, or a device that provides a cosmetic change in a subject. In some examples, an ophthalmic device may be a device that operates in a process in which vision is corrected or modified, an eye condition is enhanced or prevented, and/or through which eye physiology is cosmetically enhanced. In some examples, an ophthalmic device, or ophthalmic device components may provide optical correction, vision correction, vision monitoring, vision diagnostics, regulation of intraocular pressure, vision assistance and/or a form of ophthalmic therapy, in some examples, an ophthalmic device or ophthalmic device component(s) can refer to a contact lens, energized contact lens, soft contact lens, hard contact lens, intraocular lens, haptic, accommodating intraocular lens, a pseudo-accommodating intraocular lens, a lens, a replacement for a mammalian crystalline lens, an overlay lens, ocular insert, optical insert, implantable device, implantable telescope, subconjunctival lens, intracorneal lens, intraocular lens haptic, retinal implant or intraocular lens optic.

In some examples, an ophthalmic device or ophthalmic device component(s) may include a sensor, biosensor or a means to obtain biochemical or mechanical information. In some examples, a sensor may be configured to measure glucose concentration, oxygen concentration, electrolyte concentration, chemical analyte concentration, temperature, intraocular pressure, pulse, electrical impedance or eye movement.

In some examples, an ophthalmic device may be cosmetic. In some examples, an ophthalmic device may refer to a device that may change the appearance of the eye (e.g. iris color).

In some examples, an ophthalmic device or device component(s) may refer to a punctal plug, stent, shunt, tube, drainage apparatus, glaucoma treatment device, surgical tool, surgical tip or other implement or device implanted onto the surface of the eye or into the eye.

### III. Receiving Eye Image Data

The devices, methods and systems of the present disclosure generally include use of any suitable imaging modalities, methods, or techniques that may be suitable for generating data, or imaging data related to one or more features of a subject's first eye. In some examples, images may be received for a subject's first eye, a subject's second eye or for both eyes. Generally, images or imaging data generated by a number of eye imaging methods may be used, including but not limited to ultrasound, bio-microscopy, optical coherence tomography (OCT), tomography, magnetic resonance imaging, computed tomography (CT) scanning, light microscopy, photoacoustic microscopy, wave-front sensing, corneal tomography, scanning laser ophthalmoscopy, biometry, intraocular biometry or fundus photography.

Generally, imaging methods as described herein, may be used to image or generate imaging data of one or more features of the first eye of the subject. The one or more features of the eye of the subject generally relate to any eye anatomical feature that may be used to aid in designing and fabricating the ophthalmic device. For example, in the case of designing and fabricating a contact lens, one or more images of a subject's cornea may be generated and used for design of the ophthalmic device. In another example, the anterior chamber of a subject's eye may be imaged to generate spatial information for design of a virtual geometric model of an intraocular lens. In another example, the schlemm's canal and the cornea of a subject's eye may be imaged to generate spatial information for design of a virtual geometric model for a glaucoma stent to treat a subject's glaucoma and help alter intraocular pressure in the subject's eye. Generally, the one or more features of the subject's first eye may include but are not limited to the structures in or around the subject's eye including but not limited to crystalline lens, pupil, zonule of zinn, ciliary zonule, anterior wall segment, anterior chamber, posterior chamber, cornea, vitreous humor, vitreous body, acqueous humor, macula, corneosclera, trabecular meshwork, schlemm's canal, tear duct, corneal limbus, sclera, conjunctiva, uvea, retina, fundus, fovea, iris, ciliary body.

In some examples, the one or more images may further include measuring a distance in the x-axis of the anterior segment, measuring a distance in the y-axis of the anterior segment, measuring a distance in the z-axis of the anterior segment, determining the volume of the anterior segment, determining a partial volume of the anterior segment, imaging the anterior segment of the subject's eye, imaging the posterior segment of the subject's eye, imaging the zonules, imaging the ciliary body, and imaging the cornea. In one example, the anterior chamber of a subject's first eye may be imaged and measurements of distances in the x-axis of the anterior segment, measurements of distances in the y-axis of the anterior segment, and measurements of distances in the z-axis of the anterior segment may be determined from one or more images. These measurements may be used to generate an anatomical volume, or determine distance constraints of the anterior chamber. In some examples, measurements determined from images may be used to direct or select sizes of certain ophthalmic components. An anatomical volume may aid in the design of an ophthalmic device. In some examples, a single distance measurement may be determined from one or more images to direct lengths of one or more components of the ophthalmic device. For example, the one or more images may indicate the length dimension of a subject's eye anterior chamber. These dimensions may direct the length chosen for fabricating on or more haptic elements for an accommodating intraocular lens device.

Generally, image files may be generated by one or more imaging methods as described herein and as known in the art. Image files in one or more image formats, including but not limited to file types such as .IMG, .PIC, .PNG, .JPG, .TIFF, .GIF, etc., maybe received by a computer system. Any suitable file format may be used where the image format provides a standardized means of organizing and storing digital images. Image files, composed of digital data in one of these formats, can be rasterized for use on a computer, system, display or printer. An image file format may store data in uncompressed, compressed, or vector formats. A computer system may be used to rasterize, one or more image files received. The one or more images may be rendered as a grid of pixels, each of which has a number of bits to designate its image information equal to the image depth of the device displaying it.

### IV. Designing a 3D Virtual Model of the Ophthalmic Device

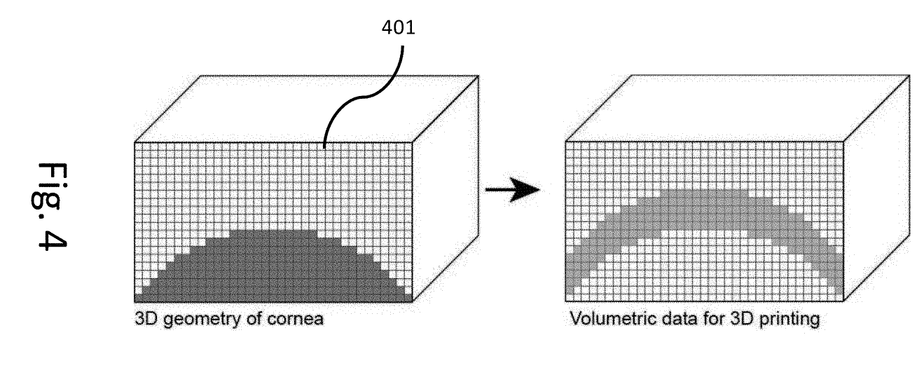

The devices, methods and systems of the present disclosure generally provide for designing a 3D virtual geometric model of the ophthalmic device using the one or more images. The one or more images used may further comprise one or more discretized imaging elements to design the three dimensional virtual geometric model of the ophthalmic device without the use of a three dimensional mathematical model based design. A three dimensional virtual geometric model includes a model constructed from information received from one or more images, in contrast to a mathematical model, whereby a virtual design may be constructed using one or more fitting equations. The devices and methods and systems of the present disclosure provide for image driven fabrication of ophthalmic devices without the use of mathematical modeling to define boundary points, lines or constraints of the model by fitting equations, or polynomial functions to generate virtual models of the device to be fabricated. The present disclosure provides for the design of geometric virtual models, whereby the boundaries and constraints of the model, as shown in FIG. 4, are designed from geometric processing of the discretized imaging elements 401, rather than being generated from a mathematical function or model. For example, if discretized imaging elements (e.g. pixels) 401 of an image are used for design, each pixel may be assigned a value during geometric processing of the image. In some examples, the values may be binary (0 or 1) and indicate the presence of feature information or absence of feature information. The presence or absence of feature information can be used to set the boundary points and lines of the geometric virtual model as shown in FIG. 4. Using the pixel 401 data, a 3D geometry of a subject's cornea 403 can be used to form volumetric data for 3D printing 405.

Generally, one or more images may comprise a set of discretized imaging elements, which may aid in design of the virtual geometric design. In some examples, a discretized imaging element is a finite piece of information bounded by space, or amount of information within a space. In some examples, discretized imaging element may be a pixel (a discretized picture element in two dimensional space) or voxel (volume pixel) in three dimensional space 401. Resolution of an image may depend on the number of pixels or voxels acquired from the one or more images of an eye feature.

A data set of one or more images of one or more features of a subject's eye may be used to construct a three dimensional representation of the feature. This information may be used to design an ophthalmic device de novo, or this information may be used to modify an existing template of a device, a precursor model or existing model of a device. For example, in the case of generating a 3D printed intraocular lens, one or more images of a subject's anterior chamber may allow for customization of a precursor haptic design. Haptic measurements of the precursor virtual intraocular device can be designed to meet the geometry and spatial constraints of a subject's individual and specific anterior chamber. In another example, a patient specific contact lens may be designed de novo without the use of mathematical modeling to calculate the curvature of the lens. In this example, corneal topography is used to assess the exact geometry of the corneal curvature from images alone, and not fitted from a mathematical function. In this example, this would allow for the virtual geometric design to more closely reflect the subject' eye feature (e.g., corneal curvature). In this example, the contact lens could be fabricated from corneal topology images alone.

Based on information of a series of discretized imaging elements, such as pixels and voxels, a 3D model can be developed. In some examples, the 3D model can be left in a form that reflects the discretized imaging elements (pixelated or voxelated). In some examples, the 3D model maybe processed further to generate smooth contours of 3D surfaces.

In some examples of the present disclosure, the virtual geometric model may be designed as a model comprising a set of pixels and voxels. In this example, this discretized information is maintained and not transformed or fitted with smoothing algorithms to generate a contoured 3D virtual model. Rather, the model remains as a set of discretized imaging elements, which are then used to direct an additive manufacturing process without the use of an intermediate fitting or smoothing step. This process may be referred to as “pixel to pixel” printing or “voxel to voxel” printing. By eliminating the smoothing and contouring step, no information is lost or gained by the model and the 3D virtual geometric model provides a highly accurate and high fidelity model of the eye feature. Since additive manufacturing may be performed with layer by layer addition, printing may be achieved by correlating individual print layers to layers of discretized elements (pixels and voxels).

In some examples, pixel to pixel to printing or voxel to voxel printing may be advantageous if the resolution of the imaging and the resolution of the additive manufacturing method (e.g., 3D printer) are high. In some cases where the resolution of imaging and the resolution of the print may not match or may be different from one another, a scaling algorithm may be applied so that the pixel/voxels of the images may be printed directly without the need for 3D contouring or fitting.

In other examples, 3D virtual geometric design may comprise applying one or more smoothing algorithms or contouring algorithms in generating the model. However, these processing elements may still be understood to be distinct from mathematically modeling algorithms that predict or inform boundary constraints of the model. In some examples, smoothing, contouring and scaling algorithms may be used to help test mechanical or structural properties of the 3D virtual geometric model.

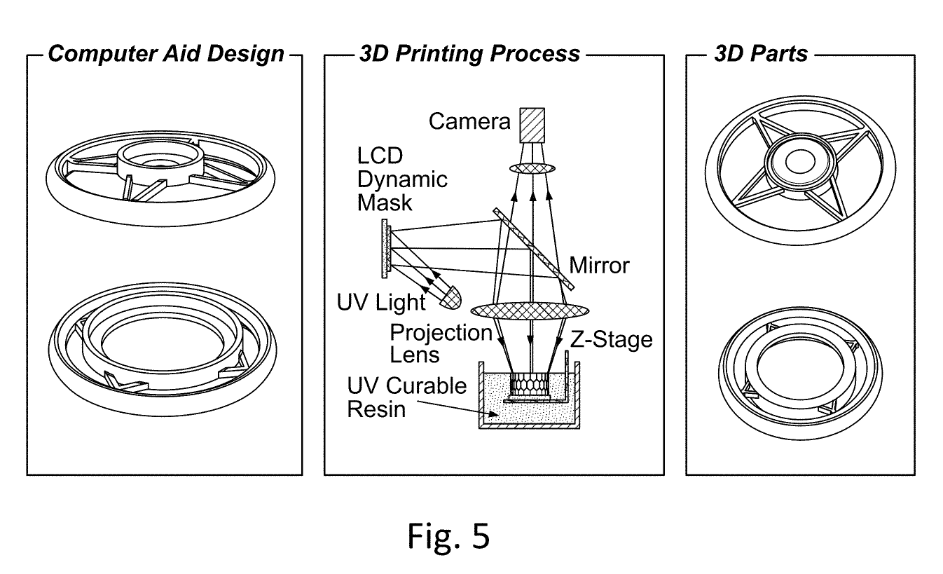

In certain examples, manipulation and design of 3D virtual geometric model may be achieved using a computer, computer system and computer aided design (CAD) software or combination thereof such as shown in the example process 500 of FIG. 5. As shown in the example of FIG. 5, a CAD design 501 of a device is formed based on acquired image data and is provided as a model in cross-sections to a 3D printing process 503. Using the cross-sectional model data, the manufacturing process 503 forms one or more 3D parts 505, such as ophthalmic device(s) fitted to a subject based on the acquired image data from the subject.

Generally, models or 3D models may be constructed using one or more computer programs, such as CAD based Solidworks. Generally any suitable 3D reconstruction methods, as known in the art, may be used in designing a model based on the one or more images received.

Generally, relative motion between consecutive images may be recovered. This process may be performed in conjunction with finding corresponding image features between these images (i.e. image points that originate from the same 3D or 2D feature). The next step may comprise recovering the motion and calibration of the camera, or imaging device and the 3D structure of the features. This process may be performed in two phases. At first the reconstruction may contain projective skew (i.e. parallel lines are not parallel, angles are not correct, distances are too long or too short, etc.). This may be due to the absence of a priori calibration. A self-calibration algorithm may be used to remove this this distortion yielding a reconstruction equivalent to the original up to a global scale factor. This uncalibrated approach to 3D reconstruction may allow much more flexibility in the acquisition process since the focal length and other intrinsic camera parameters do not have to be measured calibrated—beforehand and are allowed to change during the acquisition.

The reconstruction obtained may only include a sparse set of 3D points (only a limited number of features are considered at first). Although interpolation might be a solution, model construction may require a higher level of detail. Optionally, an algorithm may be used to match all image pixels of an image with pixels in neighboring images, so that these points too can be reconstructed. This may be accomplished by receiving the parameters of the imaging device in addition to the one or more images Since a pixel in the image may correspond to a ray in space and the projection of this ray in other images can be predicted from the recovered pose and calibration, the search of a corresponding pixel in other images can be restricted to a single line. Additional constraints such as the assumption of a piecewise continuous 3D surface are also employed to further constrain the search. It is possible to warp the images so that the search range coincides with the horizontal scanlines. This may allow use of a stereo algorithm that computes an optimal match for the whole scanline at once (Van Meerbergen et al., 2002).

Thus, depth estimations (i.e. the distance from the camera to the object surface) for almost every pixel of an image. By fusing the results of all the images together a complete dense 3D surface model may be obtained.

One or more models, or geometric virtual design, used interchangeably herein, may be generated from receiving and processing one or more images. Models may incorporate any number of features, including anatomical features that patient specific and deduced from image processing and image reconstruction from the one or more images received or pre-constructed elements that are not patient specific or combination thereof.

In some examples, a user may participate in designing a free form model based on image reconstruction performed based on receiving one or more images. In some cases, a model may be automatically generated from one or more images received. Generally, a CAD based software program may be used by a user or in an automated method to generate one or more models of the ophthalmic device.

Additionally, the present disclosure provides for the design of numerous devices whereby geometric design and mechanical function design parameters may be introduced. In some examples, a particular function or physical design parameter may be desired and introduced into the 3D virtual model geometric model to be fabricated. For example, a glaucoma stent to be placed in or near the Schlemm's canal may be designed using imaging information of the Schlemm's canal. The desired diameter of the stent may be obtained from one or more images of the Schlemm's canal of the subject's eye. However, stiffness of the stent can be selected and introduced into the 3D virtual geometric model. In this example, stiffness may be a physical parameter or comprise a functional element or functional parameter. In some examples, physical parameters can be preselected based on a desired function or selected based on geometric design. In this example, a certain flow rate for the stent may be desired. The modeled flow rate in the 3D geometric model may be achieved by altering the stiffness of the stent—a feature that may be incorporated into the 3D geometric virtual model to be fabricated.

After a 3D virtual geometric model is created from a CAD design 501, a plurality of virtual cross-sections of the 3D virtual geometric model are formed, wherein the cross-sections are defined by a set of physical parameters derived from the 3D model and are generated from the model. In some examples, the 3D cross sections correspond to a pixelated or voxelated model. In other examples the cross sections may be generated from a smoothed or processed, or contoured 3D model. The virtual cross-sections may be generated by numerous methods known in the art, such as mathematical slicing. In some examples, individual cross sections may be limited in thickness to 10 microns. In some examples, the cross-sections of the 3D model may be at least about 0.1, 0.2, 0.5, 0.75, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20, 30, 40, 50, 60, 70 80, 90, 100, 250, or 500 microns. In some examples, the cross-sections of the 3D model may be at most 0.1, 0.2, 0.5, 0.75, 1.0, 2.0, 3.0, 4.0, 5.0, 6.0, 7.0, 8.0, 9.0, 10.0, 11.0, 12.0, 13.0, 14.0, 15.0, 16.0, 17.0, 18.0, 19.0, 20, 30, 40, 50, 60, 70 80, 90, 100, 250, or 500 microns. In some examples, the cross-sections of the 3D model may range from 1-10 microns. In some examples, the cross-sections of the 3D model may range from 0.1-0.5 microns. In some examples, the cross-sections of the 3D model may range from 0.1-1 microns. In some examples, the cross-sections of the 3D model may be 1-50 microns. In some examples, the cross-sections of the 3D model may range from 5-20 microns. In some examples, the cross-sections of the 3D model may range from 10-50 microns. In some examples, the cross-sections of the 3D model may range from 25-100 microns. In some examples, the cross-sections of the 3D model may range from 50-200 microns. In some examples, the cross-sections of the 3D model may be 25-250 microns. In some examples, the cross-sections of the 3D model may be 50-500 microns. In some examples, the cross-sections of the 3D model may range from 200-500 microns.

Based on the generation of a plurality of cross sections, one or more bitmap masks may be generated to help direct additive manufacturing 503. For example, if stereolithography is used, bitmap masks may be used to help direct the use of a light source or laser to help solidify materials during additive manufacturing or 3D printing 503. A matrix of voxels may be hardened or polymerized in the material to be hardened, wherein the voxels make up a XY raster that is predetermined by the size, number and arrangement of the pixels, and the height (=hardening depth) of the voxels in the material.

In some examples, especially where stereolithography may be used, special portions of the structure to be generated—e.g., only within the cross-sectional area to be hardened—can be identified and selected. In some examples, an energy input (e.g., from a light source, etc.) can be very efficiently influenced in relation to a specific cross-sectional area—defined by its contours, or pixelated/voxelated model space—of the 3D object. In some examples, within a cross-sectional area one or more bitmaps may be used to generate one or more masks to control exposure of the material to light that may aid in the hardening or polymerization of the material.

Additionally, a voxel matrix can be generated by multiple exposures within a predetermined, defined overall image area of the building plane. An offsetting of images in the building level per cross-sectional area can be used. In some examples, offsetting images may not be necessary or may not be used. Using a voxel matrix formation, e.g., a supporting structure, overhangs and/or particularly small/filigree portions can be formed significantly finer and more accurately.

In certain examples using multiple mask exposures, the hardening depth of every single voxel can be pixel-precisely controlled so that, overall, the quality of the constructed component with regard to surface quality, compact hardness, accuracy in every detail, and tolerances can be improved, and the condition of necessary supporting structures can also be optimized.

In addition to geometry or geometric information, individual layers may be also be optimized or otherwise improved for contribution to mechanical properties in the device to be fabricated. For example, individual layers may be altered or changed during the fabrication process to produce a desired effect in the device. Changes or alterations in the mechanical properties of each layer may include but are not limited to alterations in cross-section thickness, free space coordinates, reference coordinates, shape, orientation, stiffness, hardness, strength, elastic limit, proportional limit, yield strength, tensile strength, fracture strength, ductility, toughness, fatigue ratio or loss coefficient. One or more combination of changes to these physical properties may allow for functional changes to the structure to achieve a desired function as indicated by the 3D virtual geometric model. For example, in an accommodating intraocular device including one or more bendable elements, the mechanical properties of one or more layers that make up the bendable elements may be altered to increase or decrease stiffness of the material. In some examples, the ophthalmic device may be homogenous, or approximately homogenous in the physical parameters that define the additive layers. For example, it may be desired that all layers of an ophthalmic device have essentially the same stiffness or elasticity. In some examples, the ophthalmic device may be heterogeneous, or approximately heterogeneous in the physical parameters that define the additive layers. For example, it may be desired that some layers of an ophthalmic device have a different stiffness or elasticity than other regions of device. As with bendable elements of an intraocular device, selection of physical parameters such as stiffness or elasticity may impact function (the ability to bend to further accommodating function as further described herein).

In some examples, physical parameters of one or more layers may be altered by use of one or more different materials or changes in polymerization of the device formation material, as further described herein. In some examples, physical parameters of the material may be affected by differences in exposure to an energy source (e.g., time, duration, light intensity of light exposure, etc.), exposure of certain layers to additional chemicals or additives, or exposure of layers to agents post processing (e.g., after the device has been fabricated using the additive manufacturing process.)

In some examples, the 3D virtual geometric model may be used to generate one or more molds or aids to cast an ophthalmic device 505. In some examples, the ophthalmic device 505 may be fabricated by using the plurality of virtual cross-sections of the three dimensional virtual geometric model 501 to direct an additive manufacturing method 503 to generate of one or more molds based on the three dimensional virtual geometric model 501 of the ophthalmic device 503. In some examples, the molds may then be used to fabricate the ophthalmic device using more traditional methods such as casting and stamping.

### V. Additive Manufacturing Methods

The devices, methods and systems of the present disclosure generally provide for use of any additive manufacturing method, or 3D printing method, applicable to various types or manners of producing a three-dimensional object. The building or construction may be carried out in layers (layer-wise), however may also be alternatively independent from layers. Other design options are possible. For example, the hardening process can be carried out continuously without layers; discontinuously (either with same or different or variable layer thicknesses); partially continuously and partially discontinuously (discontinuously either with same or different or variable layer thicknesses); or in a combination of various possibilities. The device and process according to the present disclosure is particularly suitable for building a three-dimensional object in a voxel matrix independent from layers or using layers. In some examples, additive manufacturing may include but is not limited to three-dimensional printing, stereolithography, microstereolithography, selective laser sintering, direct laser sintering, casting or stamping.

In some examples, the plurality if cross sections generated from the virtual geometric 3D model may be used to help guide one or more beams of electromagnetic radiation, or guide control elements controlling exposure of the device to one or more beams of electromagnetic radiation. In some examples, light sources such as lasers may be used in one or more additive manufacturing processes. For example, when stereolithography may be used, individual cross sections and corresponding masks may either direct a laser beam to harden or polymerize a specific point of material in structure at given time. In some examples, the virtual model, cross sections or masks may inform control systems to direct the laser beam directly to interact with device forming material at certain points; or direct the structure to be exposed to one or more laser beams at certain points; or direct one or more masks which may be used to selectively expose different regions/points of the device forming material.

The selective delivery of electromagnetic radiation may include an appropriate source capable of electromagnetic radiation sufficient to solidify the material to be solidified. Solidification by electromagnetic radiation may comprise a process of solidification without photoreaction, such as gelation, fusion and/or sintering. In some examples, solidification may include a process of gelation and/or solidification by photoreaction or by thermal setting reaction. Accordingly, a binding agent may be selected from the group including inert binding agents; adhesives, which may gel, solidify or cure without photoreaction or with photoreaction; and photopolymers or radiation sensitive resins, which may gel and/or solidify by photoreaction and which normally include photopolymerization, cross-linking and/or network formation processes. Additional materials not susceptible to electromagnetic radiation may also be used in conjunction with binding agents.

The device for selective delivery of electromagnetic radiation may include a mask generator for generating or projecting a mask and/or a projection unit to deliver the electromagnetic radiation selectively to the defined area or volume of material to be solidified. Electromagnetic radiation can be delivered to the building region or parts thereof by means of further suitable components, including but not limited to optical elements, lenses, shutters, voxel matrix projectors, bitmap generators, mask projectors, mirrors and multi-mirror elements and the like. Examples of suitable radiation techniques to selectively deliver electromagnetic radiation include, but are not limited to spatial light modulators (SLMs), projection units on the basis of Digital Light Processing (DLP®), DMD®, Liquid Crystal Display (LCD), Image Light Amplification (ILA®), Liquid Crystal on Silicon (LCOS), Silicon X-tal Reflective Display (SXRD), etc., reflective and transmissive LCDs, light emitting diodes (LEDs) or laser diodes emitted in lines or in a matrix, light valves, microelectromechanical systems (MEMS), laser systems, etc.

In some examples, one or more 3D printed devices may also be combined with precursor devices or devices that may not be 3D printed. In some examples, the entire ophthalmic device may be fabricated using additive manufacturing or 3D printing. In one example, an intraocular lens device may be fabricated by fabricating patient specific haptics or non lens elements and combining with a commercially available lens. In some examples, a 3D printed ring or lens placement device may be fabricated and used in conjunction with a commercially available lens including but not limited to Tecnis Aspheric (Abbott Medical Optics), AcrySof IQ (Alcon), SofPort AO (Bausch+Lomb), and Softec HD (Lenstec), Tecnis Toric (Abbott Medical Optics), AcrySof IQ Toric (Alcon), and Trulign Toric (Bausch+Lomb), Tecnis Multifocal IOL (Abbott Medical Optics) and AcrySof IQ ReSTOR (Alcon), or monovision IOLs.

### VI. Fabrication Materials

A variety of suitable material for designing and manufacturing an ophthalmic device may be used. Suitable materials may be referenced as device forming materials or polymers and may be used interchangeably herein. In some examples, material may include but are not limited to a biodegradable polymer, bio-resistant polymer, biological polymer, photosensitive polymer, an ultraviolet (UV) curable polymer, cross-linkable polymer, tunable polymer, composite, protein, biocompatible polymer, a UV sensitive reagent, a curing agent, a UV induced cross linker and a chemical catalyst, or metal. In some examples this may include but is not limited to ophthalmic compatible prepolymers which are water-soluble and/or meltable. Device-forming material may comprise primarily one or more polymers, which are in some examples in a substantially pure form (e.g., purified by ultrafiltration). Therefore, after crosslinking (e.g. by UV light exposure), an ophthalmic device may require practically no more subsequent purification, such as in particular complicated extraction of unpolymerized constituents. Furthermore, crosslinking may take place solvent-free or in aqueous solution, so that a subsequent solvent exchange or the hydration step is not necessary.

A “prepolymer” refers to a starting polymer, which can be cross-linked upon actinic radiation to obtain a cross-linked polymer having a molecular weight much higher than the starting polymer. Examples of actinic radiation are UV irradiation, ionized radiation (e.g. gamma ray or X-ray irradiation), microwave irradiation, and the like.

In some examples, polymers which may be used to form the ophthalmic device include but are not limited to polylactide, polyglycolide, polysaccharides, proteins, polyesters, polyhydroxyalkanoates, polyakelene esters, polyamides, polycaprolactone, polyvinyl esters, polyamide esters, polyvinyl alcohols, modified derivatives of caprolactonepolymers, polytrimethylene carbonate, polyacrylates, polyethylene glycol, hydrogels, photo-curable hydrogels, terminal diols, and derivatives and combinations thereof

In some examples, polymers which may be used to form the ophthalmic device include but are not limited to polyimide, Nitinol, platinum, stainless steel, molybdenum, metal, metal alloy, or ceramic biocompatible material or combinations thereof. Other materials of manufacture or materials with which the ophthalmic device can be coated, reinforced or manufactured entirely include Silicone, PTFE, ePTFE, differential fluoropolymer, FEP, FEP laminated into nodes of ePTFE, silver coatings (such as via a CVD process), gold, prolene/polyolefins, polypropylene, poly(methyl methacrylate) (PMMA), acrylic, PolyEthylene Terephthalate (PET), Polyethylene (PE), PLLA, HDDA, and parylene. The device can be reinforced with polymer, Nitinol, or stainless steel braid or coiling or can be a co-extruded or laminated tube with one or more materials that provide acceptable flexibility and hoop strength for adequate lumen support and drainage through the lumen. The implant can alternately be manufactured of nylon (polyamide), PEEK, polysulfone, polyamideimides (PAI), polyether block amides (Pebax), polyurethanes, thermoplastic elastomers (Kraton, etc.), and liquid crystal polymers. In the case of biodegradable or bioabsorbable devices, a variety of materials can be used, such as biodegradable polymers including: hydroxyaliphatic carboxylic acids, either homo- or copolymers, such as polylactic acid, polyglycolic acid, polylactic glycolic acid; polysaccharides such as cellulose or cellulose derivatives such as ethyl cellulose, cross-linked or uncross-linked sodium carboxymethyl cellulose, sodium carboxymethylcellulose starch, cellulose ethers, cellulose esters such as cellulose acetate, cellulose acetate phthallate, hydroxypropylmethyl cellulose phthallate and calcium alginate, polypropylene, polybutyrates, polycarbonate, acrylate polymers such as polymethacrylates, polyanhydrides, polyvalerates, polycaprolactones such as poly-necaprolactone, polydimethylsiloxane, polyamides, polyvinylpyrollidone, polyvinylalcohol phthallate, waxes such as paraffin wax and white beeswax, natural oils, silk protein, protein, shellac, zein, or a mixture thereof, as listed in U.S. Pat. No. 6,331,313 to Wong, which is expressly incorporated by reference in its entirety.

A solution of a device-forming material can be prepared by dissolving the device-forming in any suitable solvent known to a person skilled in the art. Examples of suitable solvents are water, alcohols, such as lower alkanols, for example ethanol or methanol, and furthermore carboxylic acid amides, such as dimethylformamide, dipolar aprotic solvents, such as dimethyl sulfoxide or methyl ethyl ketone, ketones, for example acetone or cyclohexanone, hydrocarbons, for example toluene, ethers, for example THF, dimethoxyethane or dioxane, and halogenated hydrocarbons, for example trichloroethane, and also mixtures of suitable solvents, for example mixtures of water with an alcohol, for example a water/ethanol or a water/methanol mixture.

### VII. Accommodating Lenses

The devices, methods and systems of the present disclosure also provide a device capable of accommodating function. An accommodation or accommodating function, terms used interchangeably herein, includes a process by which the vertebrate eye changes optical power to maintain a clear image or focus on an object as its distance varies.

In some examples, vertebrate eyes may vary the optical power by changing the form of the elastic lens using the ciliary body (in humans up to 15 diopters). Other vertebrate eyes may vary the power by changing the distance between a rigid lens and the retina with muscles. The devices, methods and systems of the present disclosure also provide a device capable of accommodating function or restoring accommodating function in vertebrate eyes. In some examples, the device of the present disclosure includes one or more 3D printed elements that are configured to change shape when force is applied by the eye on opposite edges of the elements and/or pressure is increased behind the ophthalmic device. In some examples, force may be applied by ciliary muscles, ciliary body or zonules or other force generating elements or features of the eye. In some examples, one or more 3D printed elements that are configured to change shape may bow or bend when force is applied on opposite edges. In some examples, one or more 3D printed elements may be haptics, or moveable or spring like elements that surround a lens. Haptics or other 3D printed elements may be configured to change shape when force is applied. In some cases changing shape may include bowing or bending. This movement may cause displacement of the lens along one or more axes. This displacement may result in changing optical power of the lens as result of the subject changing sight from near to far and thus providing accommodating function.

In some examples, the present disclosure provides for 3D printed lenses. When force is applied to these 3D printed lens devices, the lens may change shape, thus altering the optical power of the lens and allowing for accommodation. In one example, an ophthalmic lens device is configured to biomimic a natural animal crystalline lens. In this example, the device is manufactured from an elastic polymer with mechanical properties that mimic a natural animal crystalline lens. In one example, 3D printing is used to manufacture, via layer by layer process, an ophthalmic accommodating lens from a biopolymer such as silk protein or a protein polymer matrix similar to the crystalline matrix found in a natural human or vertebrate eye. In some examples, when force is applied to the lens, the lens may change shape, either becoming flatter or more round depending on the direction of the force. This change in shape may alter the optical power of the lens. In some examples, when a lens is designed to be implanted into a subject, such as a patient, the ability of the lens to change shape and optical power when forced is applied (e.g., by ciliary body or zonules) may allow restoration of accommodating function to the subject. In some examples, the intraocular accommodating lens may also be a capsular bag lens design.

### VIII. Software and Computer Systems

In various examples, the methods and systems of the invention may further comprise software programs on computer systems and use thereof. Accordingly, computerized control for the synchronization of system functions such as laser system operation, fluid control function, and/or data acquisition steps are within the bounds of the invention. The computer systems may be programmed to control the timing and coordination of delivery of sample to a detection system, and to control mechanisms for diverting selected samples into a different flow path. In some examples of the presently disclosed technology, the computer may also be programmed to store the data received from a detection system and/or process the data for subsequent analysis and display.

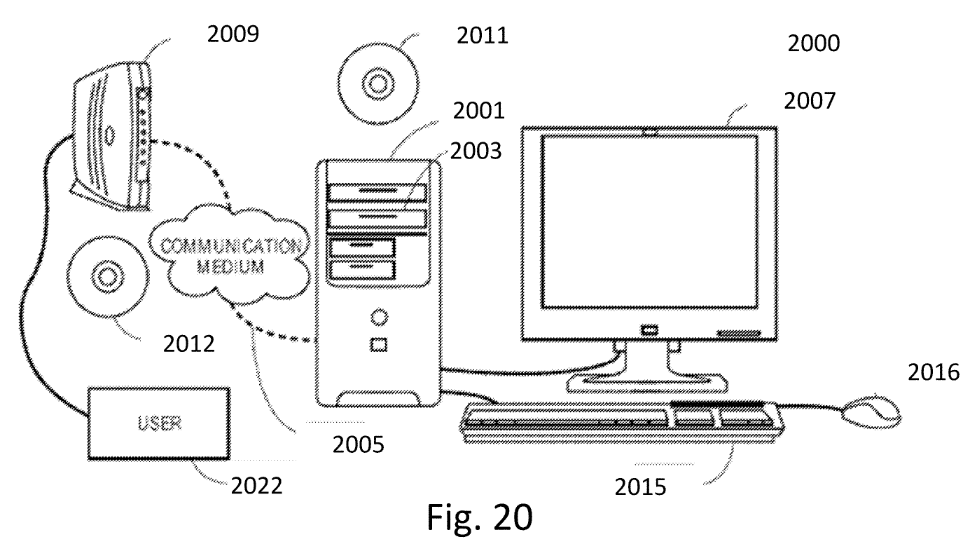

The computer system 2000 illustrated in FIG. 20 may be understood as a logical apparatus, 2007, 2005, that can read instructions from media 2012 and/or a network port, which can optionally be connected to server having fixed media 2009. The system, such as shown in FIG. 20 can include a CPU, 2001 disk drives, 2003, optional input devices such as handheld devices, 2016, for receiving one or more images, or other instrument types such as a laboratory or hospital based instrument 2011. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party or user, 2022 as illustrated in FIG. 20.

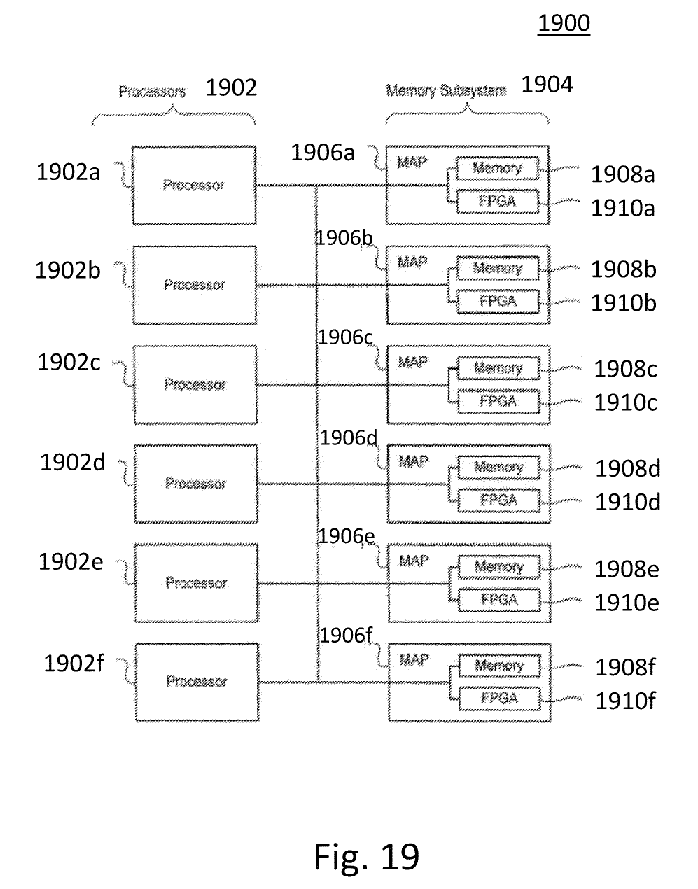

FIG. 19 is a block diagram illustrating an example architecture of a computer system 1900 that can be used in connection with example examples of the disclosure. As depicted in FIG. 19, the example computer system can include a processor 302 for processing instructions. Non-limiting examples of processors include: Intel Xeon™ processor, AMD Opteron™ processor, Samsung 32-bit RISC ARM 1176JZ(F)-S v1.O™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some examples, multiple processors or processors, 1904, 1902a, 1902b, 1902c, 1902d, 1902d, 1902e, 1902f, with multiple cores, 1906a, 1906b, 1906c, 1906d, 1906d, 1906e, 1906f can also be used, whether in a single computer system, in a cluster, or distributed across systems, 1908a, 1908b, 1908c, 1908d, 1908d, 1908e, 1908f, over a network including a plurality of computers, cell phones, and/or personal data assistant devices, 1910a, 1910b, 1910c, 1910d, 1910d, 1910e, 1910f.

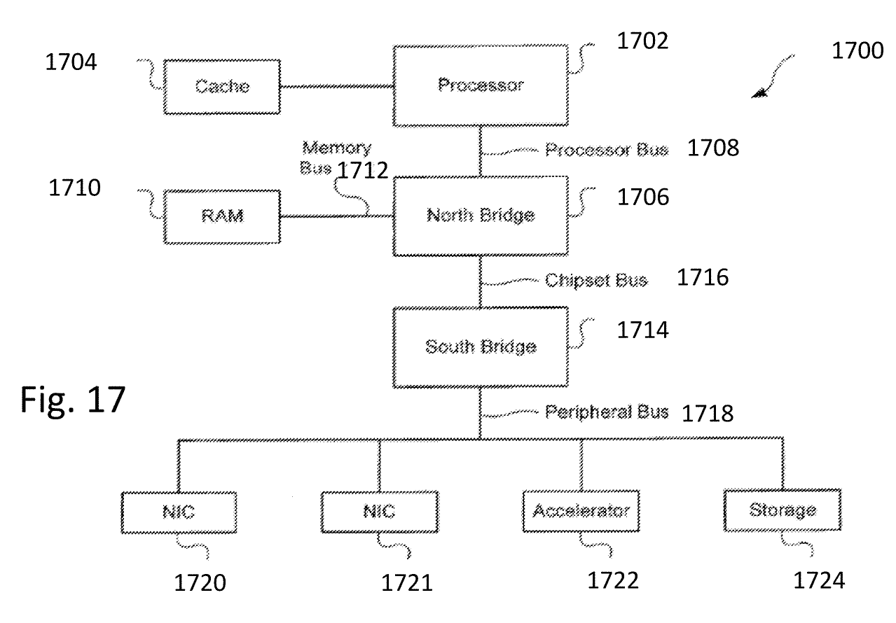

As illustrated in the example system 1700 of FIG. 17, a high speed cache 1704 can be connected to, or incorporated in, the processor 1702 to provide a high speed memory for instructions or data that have been recently, or are frequently, used by processor 1702. The processor 1702 is connected to a north bridge 1706 by a processor bus 1708. The north bridge 1706 is connected to random access memory (RAM) 1710 by a memory bus 1712 and manages access to the RAM 1710 by the processor 1702. The north bridge 1706 is also connected to a south bridge 1714 by a chipset bus 1716. The south bridge 1714 is, in turn, connected to a peripheral bus 1718. The peripheral bus 1718 can be, for example, PCI, PCI-X, PCI Express, or other peripheral bus. The north bridge 1706 and south bridge 1714 are often referred to as a processor chipset and manage data transfer between the processor, RAM, and peripheral components on the peripheral bus 1718. In some alternative architectures, the functionality of the north bridge 1706 can be incorporated into the processor 1702 instead of using a separate north bridge chip.

In some examples, system 1700, can include an accelerator card 1722 attached to the peripheral bus 1718. The accelerator 1722 can include field programmable gate arrays (FPGAs) or other hardware for accelerating certain processing. For example, an accelerator 1722 can be used for adaptive data restructuring or to evaluate algebraic expressions used in extended set processing.

Software and data are stored in external storage 1724 and can be loaded into RAM 1710 and/or cache 1704 for use by the processor 1720. The system, 1700 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example examples of the present disclosure.

In this example, system 1700 also includes network interface cards (NICs) 1720 and 1721 connected to the peripheral bus for providing network interfaces to external storage 1724, such as Network Attached Storage (NAS) and other computer systems that can be used for distributed parallel processing.

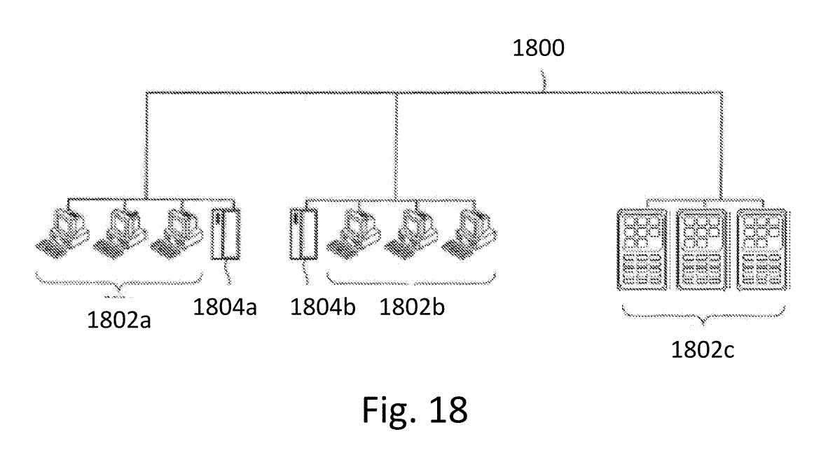

FIG. 18 is a diagram showing a network, 1800 with a plurality of computer systems 1802a, and 1802b, a plurality of cell phones and personal data assistants 1802c, and Network Attached Storage (NAS) 1804a, and 1804b. In some examples, systems 1802a, 1802b, and 1802c can manage data storage and optimize data access for data stored in Network Attached Storage (NAS) 1804a and 1804b. A mathematical model can be used for the data and be evaluated using distributed parallel processing across computer systems 1802a, and 1802b, and cell phone and personal data assistant systems 1802c. Computer systems 1802a, and 1802b, and cell phone and personal data assistant systems 1802c can also provide parallel processing for adaptive data restructuring of the data stored in Network Attached Storage (NAS) 1804a and 1804b. FIG. 18 illustrates an example only, and a wide variety of other computer architectures and systems can be used in conjunction with the various examples of the present invention. For example, a blade server can be used to provide parallel processing. Processor blades can be connected through a back plane to provide parallel processing. Storage can also be connected to the back plane or as Network Attached Storage (NAS) through a separate network interface.

In some example examples, processors can maintain separate memory spaces and transmit data through network interfaces, back plane or other connectors for parallel processing by other processors. In other examples, some or all of the processors can use a shared virtual address memory space.

The above computer architectures and systems are examples only, and a wide variety of other computer, cell phone, and personal data assistant architectures and systems can be used in connection with example examples, including systems using any combination of general processors, co-processors, FPGAs and other programmable logic devices, system on chips (SOCs), application specific integrated circuits (ASICs), and other processing and logic elements. In some examples, all or part of the computer system can be implemented in software or hardware. Any variety of data storage media can be used in connection with example examples, including random access memory, hard drives, flash memory, tape drives, disk arrays, Network Attached Storage (NAS) and other local or distributed data storage devices and systems.

In some examples of present disclosure, the computer system can be implemented using software modules executing on any of the above or other computer architectures and systems. In other examples, the functions of the system can be implemented partially or completely in firmware, programmable logic devices such as field programmable gate arrays, system on chips (SOCs), application specific integrated circuits (ASICs), or other processing and logic elements. For example, the Set Processor and Optimizer can be implemented with hardware acceleration through the use of a hardware accelerator card, such as accelerator card.

### IX. Terminology

The terminology used therein is for the purpose of describing particular examples only and is not intended to be limiting of a device of this disclosure. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Several aspects of a device of this disclosure are described above with reference to example applications for illustration. It should be understood that numerous specific details, relationships, and methods are set forth to provide a full understanding of a device. One having ordinary skill in the relevant art, however, will readily recognize that a device can be practiced without one or more of the specific details or with other methods. This disclosure is not limited by the illustrated ordering of acts or events, as some acts may occur in different orders and/or concurrently with other acts or events. Furthermore, not all illustrated acts or events are required to implement a methodology in accordance with this disclosure.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another example. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5.

### VIII. Examples

### Example: Patient Specific Contact Lens

A high resolution Projection Micro-stereolithography system (PμSL) was used to fabricate a customized contact lens. This example provides for patient-specific additive manufacturing to create a personalized contact lens, fitted precisely to a subject's cornea. Custom fit contacts may improve a subject's visual acuity when a customized contact lens can accommodate for imperfection in the cornea curvature, especially suffering from astigmatism, keratoconus, or irregular cornea shapes. Corneal pachymeter, which uses ultrasound and the Scheimpflug principle to measure the thickness and topography of the cornea, can be used to image a subject's cornea to personalize the lens shape of a contact lens device to be manufactured by 3D printing.

The contact lens was designed using a computer aided design (CAD) model of a stereolithography (STL) file which was sliced into 2D layers of bitmap image files for the dynamic mask to project. The STL file was designed with Solidworks and the images were created with MATLAB for example. The program automatically generated images of the horizontal plane of the structure at each layer in black and white bitmap files.

UV light for stereolithography was produced with Mercury I-line emission, and the UV light used had a 365 nm wavelength. The UV light intensity was controlled by the current, and generally ranged from 400 mA to 800 mA, where UV intensity increased linearly with the current from 3.43E-5 W to 5.89E-5 W respectively. For process reliability, the light illumination was kept uniform with use of a collimating lens to straighten the UV light, and at the mercury output, the fly-eye type optical homogenizer was installed to maintain the intensity variation within 5%. UV light was directed through the LightGate prism, reflected to a Digital Micromirror Device (DMD), and then directed back with the image made by the DMD.

DMD was used as the dynamic mask that provided the image from the sliced CAD model. With DMD, the UV illumination had 88% reflection modulation efficiency, so 12% of the UV intensity was lost. DMD had 1400× 1050 pixel arrays, each pixel having 13 μm×13 μm size. The UV light was reflected back at the white part of the bitmap image, and was not reflected at the black part. The reflected UV projection from DMD was then focused with the projection lens, and reflected to the resin surface for photopolymerization. The modulated light from DMD chip was transferred through a reduction lens (same as the projection lens), so the image on the resin surface had a reduced feature size compared to the DMD defined mask pattern. The 13 μm pixel size of DMD chip was projected onto the resin surface as 7.1 μm pixels. Therefore, the maximum horizontal plane printable size was 9.940 mm×7.455 mm and was theoretically unlimited vertically.

Silicon wafer was used for the platform to fabricate the 3D structure. It moved vertically with the computerized mechanical translation stage with a translation precision of 0.1 μm. For focusing the platform at the right position, the UV light reflected back from the silicon wafer was directed back to the minor, projection lens, beam splitter, and then the camera to visualize the image on the wafer. With the camera and the displacement sensor, the platform was focused and aligned at the focal length of the projection lens, and then the top of the resin was aligned with the platform. During the fabrication process, the platform was dipped inside the resin 1 mm, then brought back up until the top of the fabricated sample was 20 μm below the resin surface; for each step the platform went 1000 μm down and 980 μm up. 20 μm was the minimum thickness for PμSL using HDDA (1,6-hexanedioldiacrylate) as polymer material.

After the mechanical movement of the stage, the resin on the top of the platform or the sample in process was UV polymerized as the image reflected from DMD. The time to polymerize a single layer varies with the polymer, thickness, and UV intensity. In this example, using HDDA polymer and using 400 mA current for 20 μm thick layer, the curing time was 12 seconds.

UV curing polymer used in the PμSL system was HDDA (1,6-hexanedioldiacrylate), which was a low viscosity monomer that can be cross-linked with UV light with the help from photo-initiator. The photo-initiator used was Irgacure 819 (Bis(2,4,6-trimethylbenzoyl)-phenylphosphineoxide) from BASF. Irgacure 819, whose absorption spectrum shows that it works well in 365 nm wavelengths, was a photoinitiator used. In some examples, UV inhibitor may be needed to control curing depth and to reduce the unnecessary curing regions from the UV light source and external lights. Without the inhibitor, the light may go all the way down to the bottom of resin container and UV light may cure resin along its vertical path. Also, because of the diffractive nature of light, Gaussian distribution of diffractive light may be expected to spread over every single pixel. In some examples, if the resin is sensitive to UV light and is easily polymerized at the unwanted Gaussian distribution region, the 3D model may not have high resolution nor be accurate. Sudan I (1-phenylazonaphth-2-ol), 4-Allyloxy-2hydroxybenzophenone and 2-(2H-Benzotriazol-2-yl)-6-dodecyl-4-methylphenol (Sigma Aldrich, named by BASF as Tinuvin 171) were used as UV absorbers. Sudan I was essentially an orange-red solid dye traditionally used in food industry. Because of orange-red color, Sudan I absorbs UV in 364 nm wavelength, but because of that, the resin and lens itself becomes orange-red in color. 4-Allyloxy-2hydroxybenzophenone was a type of hydroxybenzophenone, well known chemical for UV stabilizing neutral or transparent applications at 330-370 nm range. 2-(2H-Benzotriazol-2-yl)-6-dodecyl-4-methylphenol was a liquid UV absorber of the hydroxyphenylbenzotriazole class. Tinuvin 171 was also used in this example.

For the resin used in the PμSL, 97 wt % of HDDA (Sigma Aldrich) was mixed with 1 wt % Irgacure 819 (BASF) and 2 wt % Tinuvin 171 (Sigma Aldrich). The HEMA solution included 63% HEMA (Sigma Aldrich), 1% EGDMA (Sigma Aldrich) and 35% deionized water, and 1% Irgacure 1173 (BASF).

Because additive manufacturing fabricates a structure layer by layer, one result of 3D printing is the stair-stepping effect. This stair-stepping effect was reduced by many different methods. First, the stair-stepping effect depends on the layer thickness. Because the size of the stair is proportional to layer thickness, having thin 2D layers can reduce the effect.

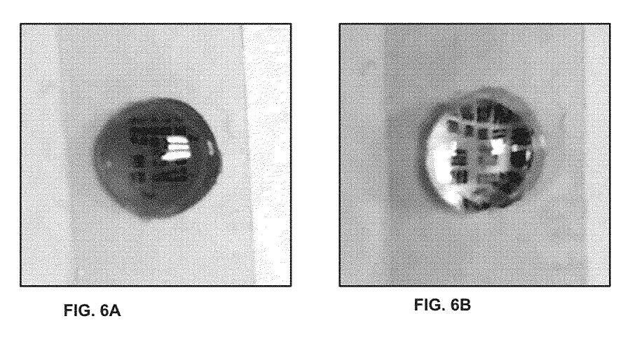

To make an optical lens, the stair-stepping effect was reduced until light does not diverge at the stair-stepping effect region. This was tested by performing air force target testing with 1951 USAF resolution test chart (a resolution test pattern set by the U.S. Air Force in 1951) as shown in FIGS. 6a and 6b. Lenses from additive manufacturing may be made with different post-processing methods. Sanding is an effective and inexpensive way to smooth the surface. The parts can be sanded by hand or with sanding belt machine. Belt sanding is fast, but may be limited by ability to control the smoothing process. Bead blasting can also smooth the surface by blasting small particles. However, this must be processed by human hand, therefore cannot be mass finished. Another method is vapor smoothing. The printed object is dipped into a boiling vapor tank, which can melt the surface. Use of vapor smoothing methods on samples may also require additional smoothing steps, including polishing.

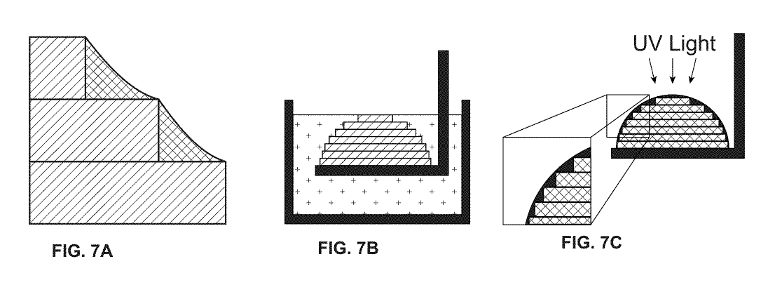

Also, by continuously generating monolithic polymeric parts and not by stacking layers one by one, the stair-stepping effect was reduced. Continuous liquid interface could be done with the “dead zone” oxidation where photo-polymerization is inhibited by oxygen between the window and the polymerizing part to avoid polymerization on the window surface. Meniscus modeling can be used for reducing stair-stepping effect. After the layer-by-layer fabrication, the steps were filled layer-by-layer with the same UV curable material. Because of the surface tension, viscosity and different shapes of the printed parts, the shape of meniscus filling may vary as shown in FIGS. 7a-c.

By wetting the polymerized horizontal surface of one layer and the polymerized vertical surface of the next layer with the liquid resin, the resin created a meniscus shape between the layers, because of the resin surface tension. With this lens fabrication, example the whole surface of layer-by-layer lens is wetted with the resin, and then the whole lens was UV polymerized as shown in FIGS. 7a-c. FIG. 7a is schematic diagram of wetting a 3D printed device 701 with curable material 703. FIG. 7b is schematic diagram of dipping a 3D printed device 705 in curable material 707. FIG. 7c is a schematic diagram of curing or polymerizing curable material 709 during a post processing reaction 711 to smooth 3D printed layers 713.

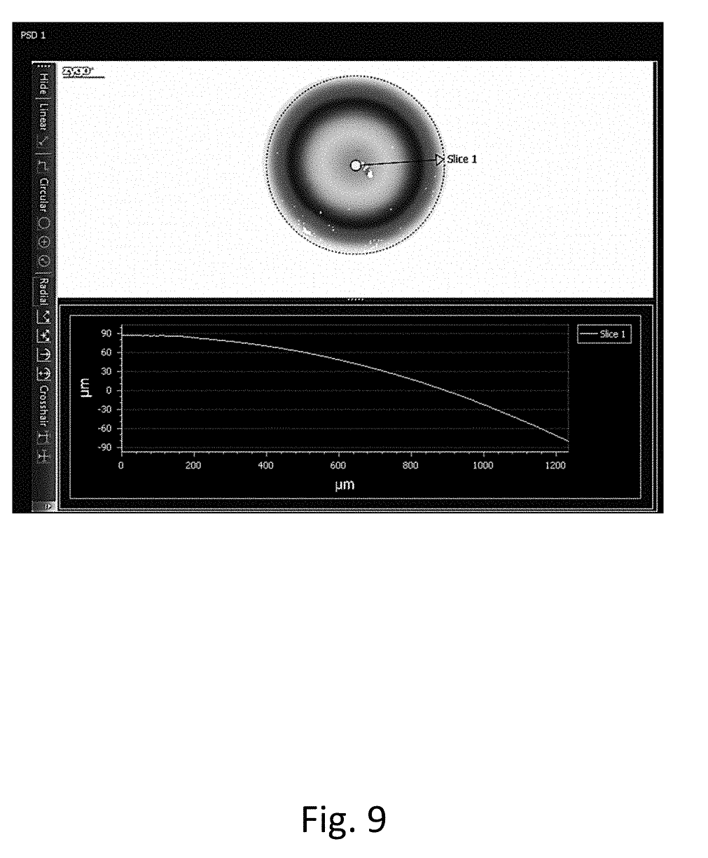

The shape of the lens was drawn with Solidworks as shown in the example of FIG. 9. The cylinder shaped platform was first built to have a firm, stable and flat surface, then a circular dome shape was drawn, top of the platform as seen in The designed diameter of the platform is 7 mm and the height of the dome is 2 mm. Because the dome was in circular a shape, the radius of the dome circle is 4.0625 mm. Using Zygo 3D Optical Surface Profiler, the outer shape of the printed lens was characterized. The lens with stair-stepping effect had the radius as ˜˜mm and the lens with reduced stair-stepping effect had the radius as ˜˜mm.

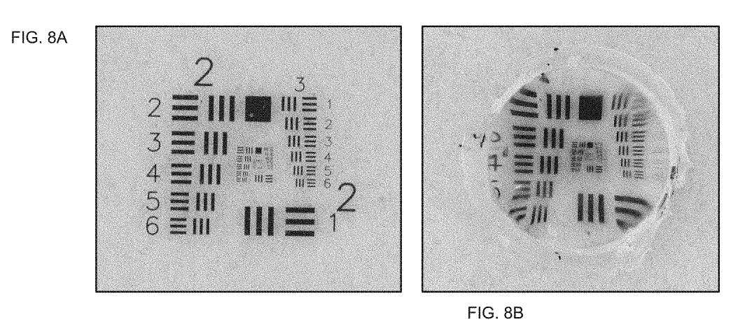

To demonstrate the capability of a convex lens, an image was put right below the lens to see how the lens changed. FIG. 8a is an image of Air Force target testing of an example 3D printed lens post processed with resin curing. FIG. 8b is an image of a 3D printed lens on top of the Air Force target. The USAF 1951 Chart test target was used in this experiment to see if the lens could show the test target with the similar resolution. The USAF 1951 chart test has Groups, each of which have 6 elements each. The purpose of this test was to visualize which element in the Group and determine the last one to show non-blurry separate bars. The picture in the example of FIG. 8b shows clear separate lines until Group 4 Element 5. With the lens directly printed from the PμSL on the test target, the last visible bars are in Group 4 Element 4.

### Example: Patient Specific Contact Lens via 3D Printed Mold

This example provides methods for fabricating and prototyping patient specific contact lenses using 3D printed molds. To have a material to be used for the contact lens, the material has to be biocompatible, hydrophilic and transparent. HEMA (2-Hydroxyethyl methacrylate) is a well-known hydrogel polymer used for contact lens. To make HEMA cure in UV light, large amount of HEMA was mixed with ethylene glycol dimethacrylate, deionized water, and UV initiator.

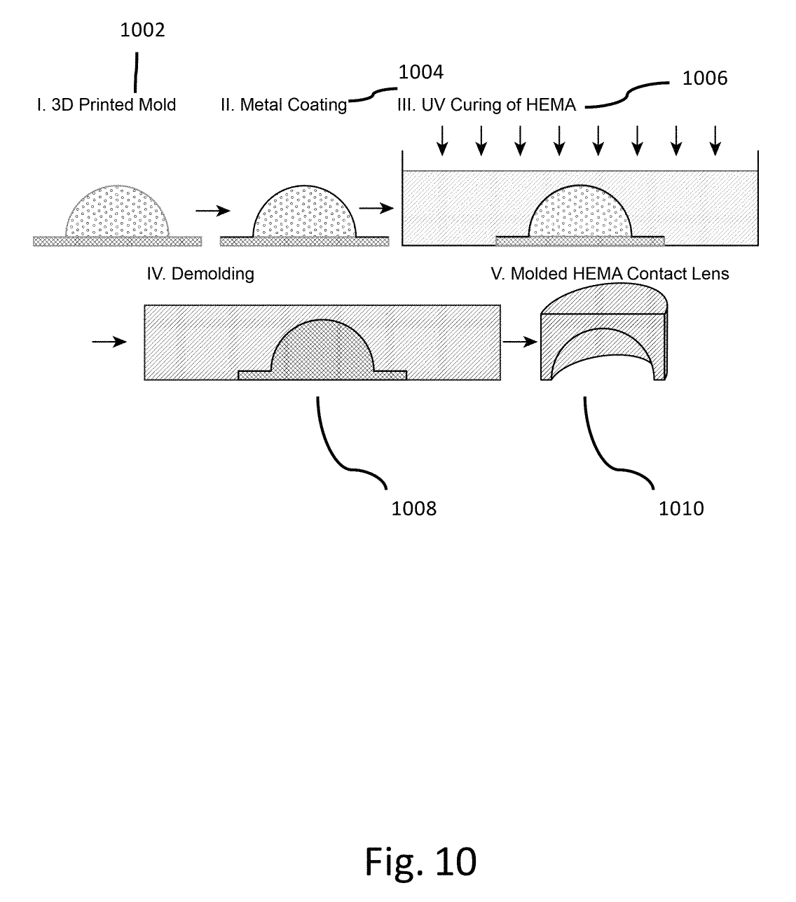

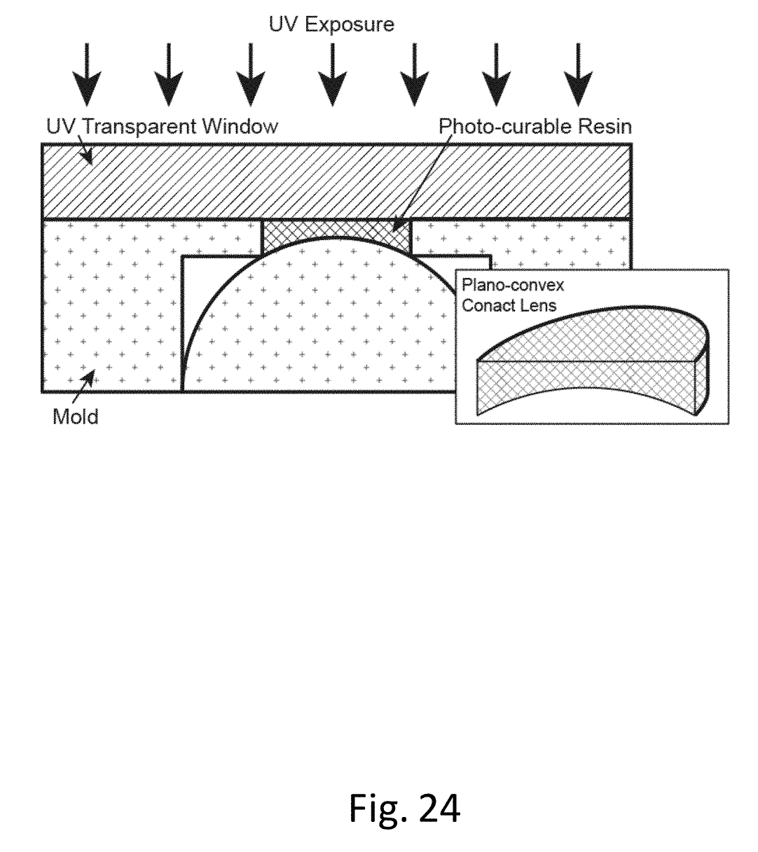

As shown in the example of FIG. 10, the 3D printed mold 1002 was coated with Au—Pd alloy, 1004, using the sputter coater. Without the metal coating, when HEMA was polymerized on top of the HDDA mold, 1006, the HEMA mold firmly sticks to the HDDA mold 1008. The mold 1008 was put inside the petri dish, and the fixed amount of HEMA was poured inside for uniform thickness. The petri dish was then UV polymerized in a UV oven for 5 minutes. Then the polymerized HEMA was taken out and cut into a lens shape 1010. With this method as also shown in the example of FIG. 24, the lens had a planar concave shape.

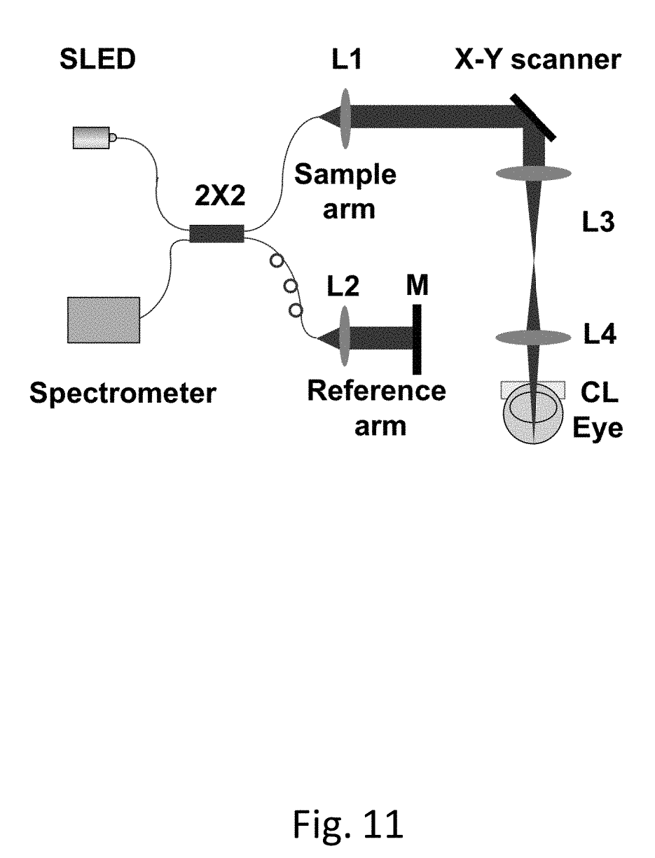

As described above, FIG. 3 shows the schematic of the experimental system, 319. In certain examples, an optical coherence tomography (OCT) system, such as the example OCT system 1100 illustrated in FIG. 11, can be used. The example system 1100 used a broadband Superluminescent LED (SLED) 1102 for illumination (center wavelength: 840 nm; bandwidth: 95 nm; IPSDW0825C-0314, Inphenix). The illumination beam was first coupled into a 50×50 fiber coupler (OZ optics) 1104 and then split into the sample arm 1106 and reference arm 1108. In the sample arm 1106, the collimated OCT probing beam reached a pair of galvanometers (QS-7, Nutfield Technology) and entered the pupil after two lenses (L3 and L4). A customized spectrometer 1110 collected interference signals between the sample arm 1106 and reference arm 1108. The images were captured in 50 kHz A-line rate with a 256×256-pixel resolution.

Pigmented rats were imaged (e.g. 250-g Sprague Dawley and Long Evans rats; Charles River Laboratories). During experiments, animals were anesthetized by a mixture of isoflurane and normal air (2% isoflurane at 3 liter/minute for 10 minutes and 1.5% at 2 liter/minute in following experiments). The rat eyes were anesthetized using a drop of 0.5% Tetracaine Hydrochloride ophthalmic solution and dilated using a drop of 1% Tropicamide ophthalmic solution. During experiments, rats were placed on a homemade animal holder. Before applying the contact lens to the animals, two drops of artificial tear was added on both the cornea and contact lens. No artificial tear was added after wearing contact lens.

### Example: Patient Specific Accommodating Intraocular Lens (IOL)

This example provides methods and systems for fabricating and prototyping patient specific accommodating intraocular lenses. Different accommodative IOLs (AIOLs) have been developed to restore accommodation by transmitting ciliary-muscle contractions into a change in refractive power of the eye. These AIOLs aim to restore good vision without compromising distance vision (and fewer optical side effects). The available designs include (1) single-optic AIOLs, such as the Ring-haptic Bio-ComFold IOL, ICU IOL, CrystaLens, and KH 350 IOL; (2) dual-optic AIOLs, such as the Synchrony accommodating IOL; and 3) IOLs that alter the shape and thus refractive power, such as Nu-Lens accommodating IOL. However, due to anatomical differences between patients and variability in patient's reaction to existing IOLs, even the latest and only FDA-approved accommodating lens, Crystalens, has shown inconsistent effectiveness, sometimes failing to flex, tilting of the IOL in the eye, and resulting in astigmatism and vision loss.

To overcome the limitations of the existing AIOLs described herein, the example provides for a method of designing and fabricating novel 3D printed AIOLs tailored specifically for individual patient anatomies. The functions of AIOL can be categorized as (1) the lens for projecting optical images on retina and (2) haptic to transduce the ciliary muscle contraction into the desirable anterior displacement of the lens optic, in order to accommodate the vision at near distance mimicking accommodation to focus near objects on the retina. In some examples design focus may be on the haptic for its critical role in determining the accommodation characteristics. In others it may be on the lens (e.g. multifocal and toric lenses)

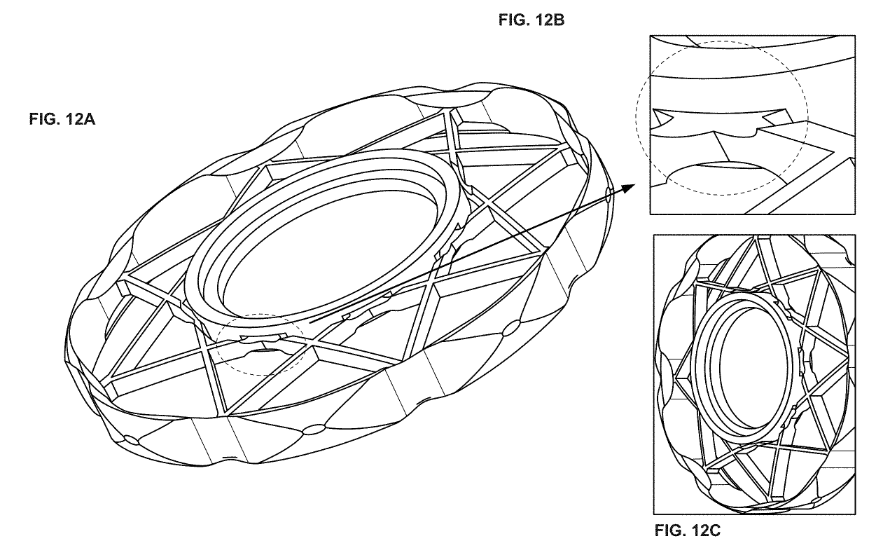

FIG. 12a is an image of an example 3D printed accommodating intraocular lens design in CAD software. FIG. 12b is a magnified image of a functional element of the example 3D printed accommodating intraocular lens design. The element shown in FIG. 12b is a stress joint that allows movement of the cross bars when force is applied. FIG. 12c is an image of 3D printed device, printed from the example design shown in FIG. 12a and FIG. 12b.

The haptic illustrated in FIGS. 12a-c was designed to include an outer mounting ring and an inner optical ring being connected by the mechanical supporting beams. The features of this haptic design are: (1) the shape of the mounting ring is tailored to fit the inner geometry of capsular bag of the specific patient to allow for the perfect fit; (2) lens ring allows for insertion of a large optic with the diameter of 6 mm to avoid the side effects such as halos and glare; and (3) the supports of the optic ring with specifically designed geometry to facilitate preferential anterior displacement of the lens and also eliminates the tilting of the lens found with the Crystalens. In addition, this design was generated to help mitigate the collapse of the capsular bag by keeping the anterior and posterior capsules apart. The geometry and the materials properties can be tailored to provide a precise accommodation for each individual patient.

Another aspect of this design is that this AIOL could be fabricated for surgical implantation of the IOL as a two-stage process after the cataract is removed. The haptic is implanted and then its position is verified with intra-operative biometry to confirm the power of the optic to be placed in the lens ring so the lens can be adjusted as needed. However, such a new design concept requires customized components that is not compatible with traditional mass-production methods such as injection molding process.

To fabricate this device, a common polymer for biomedical devices was chosen for device forming material. Among a wide range of polymer materials being used to construct IOLs, Hydroxyethylmethacrylate (HEMA) and their derivatives represent a large family of FDA approved biocompatible materials that have been widely used for preparing medical devices and implants. As an example, poly-HEMA hydrogels are flexible and transparent, therefore commonly used in producing contact lenses and intraocular lenses. When it is polymerized, although it is hydrophobic, when it is swelled in water, it can absorb water up to 600% of its own weight. Poly-HEMA was a unique choice for two reasons: (1) proper hydrophilic/hydrophobic balance and produced hydrogels with suitable mechanical properties for IOL applications; and (2) its optical properties changed only slightly with changes in the concentration of crosslinking agent.

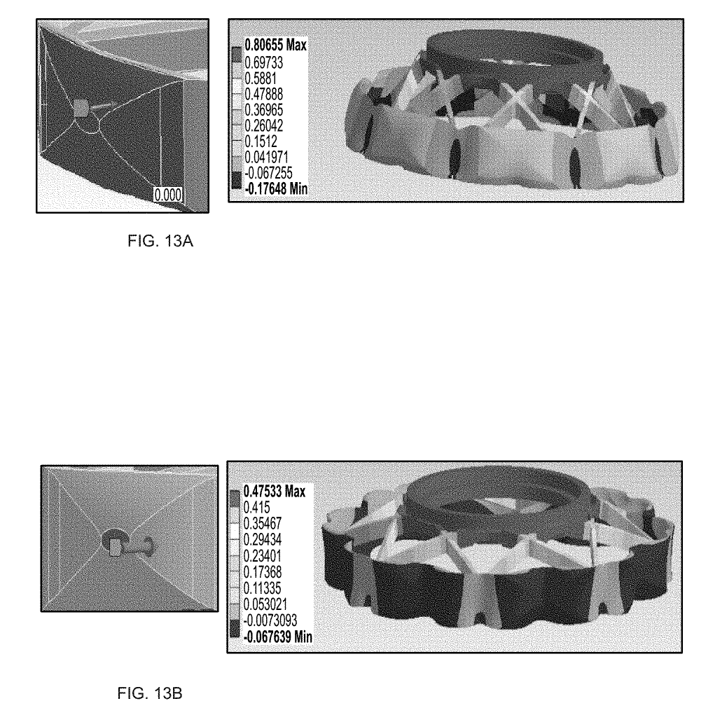

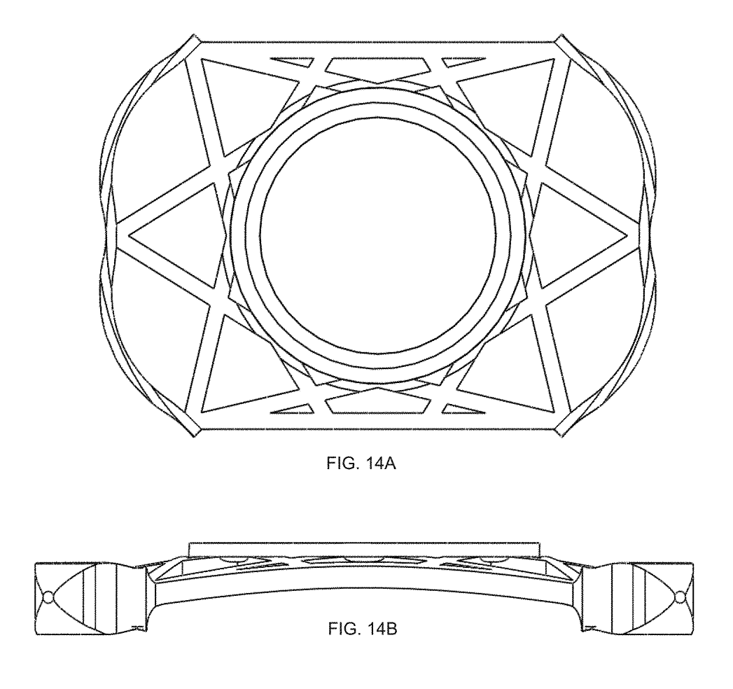

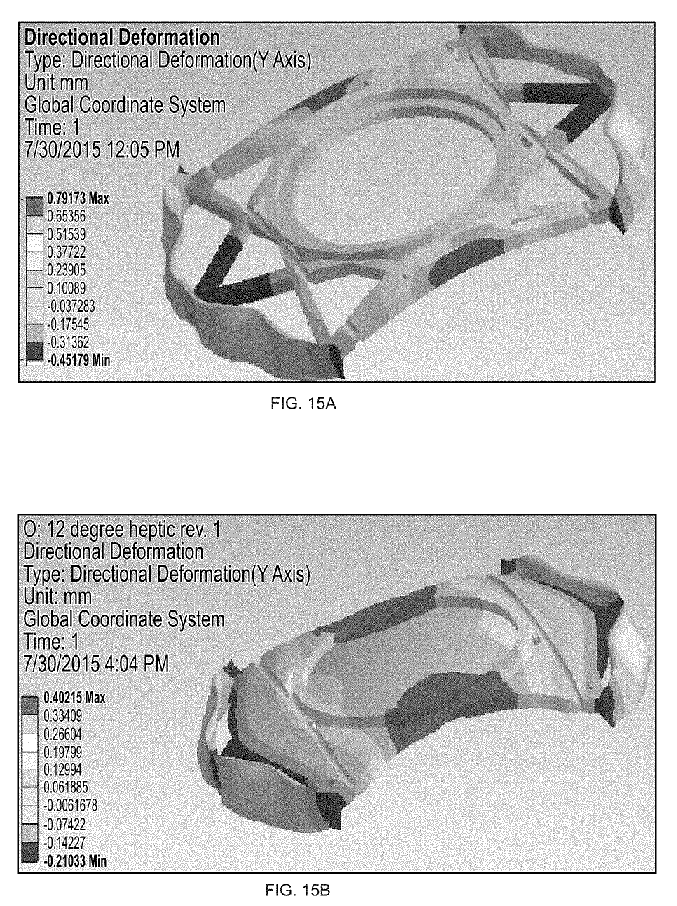

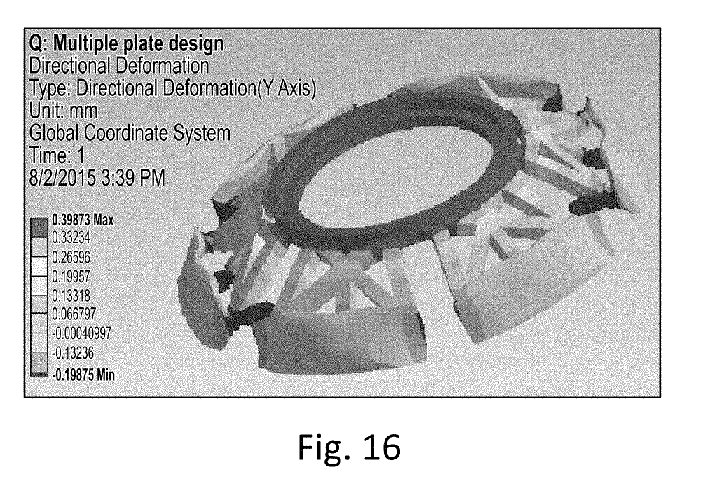

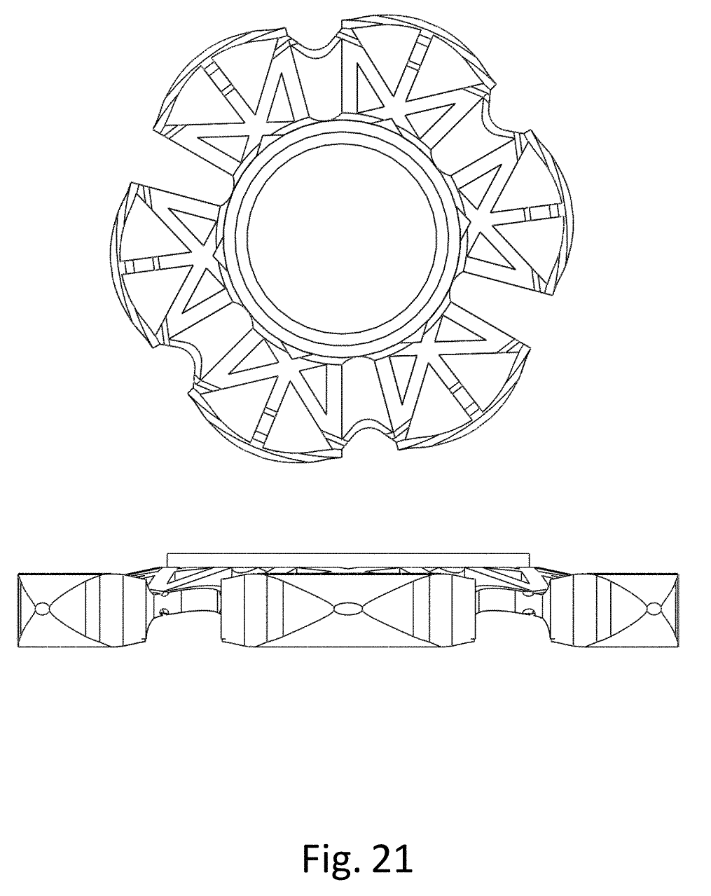

Typical haptic designs have two arcs on opposite sides of the lens. These arcs, when IOL is inserted into the capsular bag, extend outward to establish the force contact with ciliary muscle and thus, transduce the displacement induced by ciliary muscle contraction. Based on the conceptual design illustrated in FIGS. 13a and b, we further incorporated a sinusoidal pattern in the mounting ring to increase its compliance and thus, to increase the induced anterior displacement of the lens mount. Our design was then created using the commercial CAD software (Solidwork) and analyzed with commercial finite element analysis (FEA) software (ANSYS). We applied an even distributed force along the mounting ring. According to previous study by Fisher, the maximum force that the ciliary muscle exerts is 11.74 mN. We utilized this value for our analysis. From the FEA, we were able to determine that this design will allow the lens holder section a maximum displacement of 0.95 mm, which corresponds to the a single-optic accommodating IOL of power of +19 Diopters producing a change of approximately 1.14 Diopters. FIGS. 12a-c show the example CAD drawing of our part (side and isometric view FIG. 21a) as well as the ANSYS 15 analysis of our part (top and side views FIG. 13). The ANSYS images represent the deformation in the Z-axis. Additional designs were also constructed as shown in FIGS. 14a-b. FIGS. 15a-b show force simulation of different aspects of the IOL design including which parts of the device have the greatest capability for displacement or movement. FIG. 16 shows another alternative design whereby the design incorporates a feature that the lens can be folded about one or more axes during implantation into a human subject or patient. FIGS. 21a-b show atop (FIG. 21a) and side (FIG. 21b) view a foldable accommodating intraocular lens.

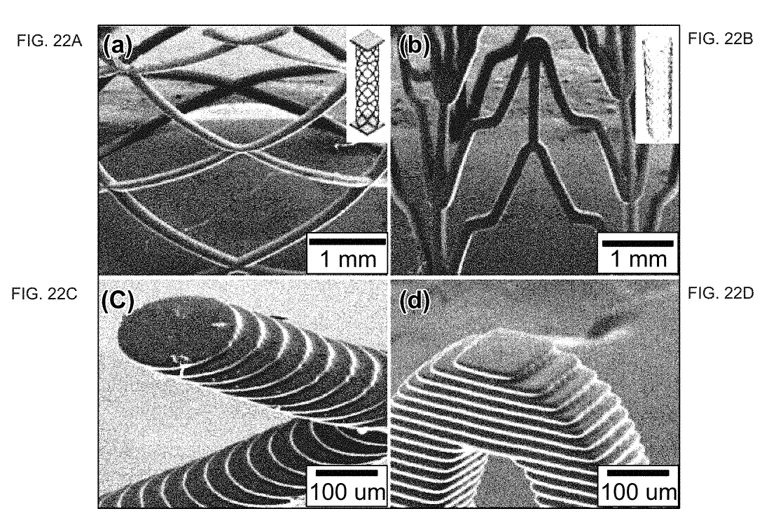

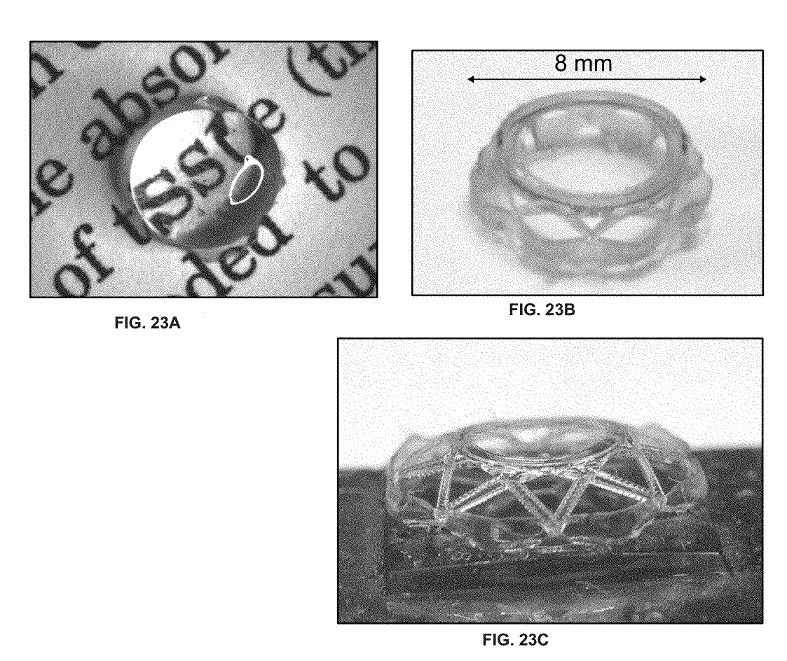

3D Printing of AIOLs: The structures shown in FIGS. 22a-d and 23a-c, were fabricated using the high-resolution 3D printing process PμSL. Cross sections of the virtual geometric design were sliced using the CAD model into a series of bitmap images. These bitmap images represent the cross-section of our structure. This conversion was done using a customized Matlab program. With the Matlab program, we can determine cross section thickness. To ensure the dimensional accuracy, the AIOL Haptic shown in FIG. 23b was fabricated over 900 layers with each layer of 20 um thick. The actual fabrication of the part took place within a small open container of resin. This resin was UV photocurable. The cross section bitmap images were projected onto the resin surface using a LCD dynamic mask. The curing of the section was controlled by the intensity of the light and the amount of exposure time. Due to the extremely parallel nature of the projection based printing process, the whole haptics was printed in less than an hour. The device was printed onto a silicon wafer in a layer-by-layer fashion as the typical additive manufacturing process. After the whole structure was completed, the wafer was removed and printed structure from the resin container. After post-process cleaning, high-resolution 3D printed IOL designs were obtained. FIG. 23a is an illustrative example a 3D printed lens without additional non-optical components such as haptics. FIG. 23b is an illustrative example of a 3D printed haptics ring that may be used with a commercially available lens. FIG. 23c is an illustrative example of a 3D printed IOL containing both a haptics elements and a lens element.

### Example: Optimization of Biocompatible Materials for 3D Printed Ophthalmic Devices

In particular, poly-HEMA cross-linked with ethylene glycol dimethacrylate (EGDMA) forms the basis of many types of daily wear soft contact lenses. Recent study suggested that with the addition of the photo-initiator, polymerization of the poly(HEMA)-EGDMA copolymer can be initiated upon the exposure of the ultraviolet (UV) light. In this example, a photo-curable resin consisting of 63% HEMA, 1% EGDMA, 35% deionized water as the solvent, and 1% Irgacure 1173 as the photo-initiator was tested and optimized for producing 3D printed soft contact lenses. Using the apparatus illustrated in FIG. 3, the photo-curable resin confined within the metallic mold and the UV transparent windows were polymerized upon the exposure of 100 W UV light for 5-10 min and subsequently released to produce the contact lens with plano-convex geometry. The use of the stainless steel sphere allows the flexibility to adjust the curvature of the convex surface to better fit the geometry of the cornea of the test subject, which in this case the rat eye.

Additional tests have been performed to characterize the key characteristics of the photo-polymerized hydrogel material, including refractive index, optical transmission, Young's modulus, and the contact angle measurement to quantify its hydrophilic properties. In this study, the refractive index of the hydrogel is measured to be 1.43. The optical transmission of 97% over the visible spectrum for 0.5 mm thick hydrogel film was observed. Slightly lower transmission at the short-wavelength range is possibly due to the absorption by the photo-initiator being added. It can be the potential subject of investigation for the further improvement. The Young's modulus (E) was measured in a cylinder compression test using a 1 in thick cylindrical sample of hydrogel. The measured value of E=1.48 MPa is in comparable with the Young's modulus found in the commercial contact lenses. Finally, the hydrophobicity test was performed by placing a water droplet (2 μl) on our sample of hydrogel with the measured contact angle of 40.46°, validating hydrophobic nature of the hydrogel.

In summary, certain examples have successfully validated the feasibility in further developing the FDA approved biocompatible materials into photo-polymerizable form but still preserve its key optical, mechanical, and chemical characteristics, while the sensitivity of photo-curable resin is yet to be optimized.

What is claimed is:

1. A method for generating an ophthalmic device for a first eye of a subject, the method comprising:
a. receiving one or more images of one or more features of the first eye of the subject;
b. designing a three dimensional virtual geometric model of the ophthalmic device using the one or more images;
c. generating a plurality of virtual cross-sections of the three-dimensional virtual geometric model, wherein the cross-sections are defined by a set of physical parameters derived from the three-dimensional model;
d. fabricating the ophthalmic device using the plurality of virtual cross-sections of the three dimensional model to direct an additive manufacturing method.

2. The method of claim 1, wherein the receiving one or more images of the one or more features of the eye of a subject further comprises use of ultrasound, bio-microscopy, optical coherence tomography, tomography, magnetic resonance imaging, computed tomography scanning, light microscopy, photoacoustic microscopy, wave-front sensing, corneal tomography, biometry, intraocular biometry or fundus photography to generate the one or more images.

3. The method of claim 1, wherein the designing a three dimensional virtual geometric model of the ophthalmic device using the one or more images further comprises receiving one or more discretized imaging elements to design the three dimensional virtual geometric model of the ophthalmic device without the use of a three dimensional mathematical model based design.

4. The method of claim 1, wherein the one or more features of the first eye of the subject further comprise a crystalline lens, pupil, zonule of zinn, ciliary zonule, anterior wall segment, anterior chamber, posterior chamber, cornea, vitreous humor, vitreous body, acqueous humor, macula, corneosclera, trabecular meshwork, schlemm's canal, tear duct, corneal limbus, sclera, conjunctiva, uvea, retina, fundus, fovea, iris, ciliary body, measuring a distance in the x-axis of the anterior segment, measuring a distance in the y-axis of the anterior segment, measuring a distance in the z-axis of the anterior segment, determining the volume of the anterior segment, determining a partial volume of the anterior segment, imaging the anterior segment of the subject's eye, imaging the posterior segment of the subject's eye, imaging the zonules, imaging the ciliary body, and imaging the cornea.

5. The method of claim 1, wherein the designing a three dimensional model of the device further comprises modifying a precursor virtual geometric model of the ophthalmic device using the one or more images of one or more features of the first eye of the subject.

6. The method of claim 1, wherein the set of physical parameters further comprises cross-section thickness, free space coordinates, reference coordinates, shape, orientation, stiffness, hardness, strength, elastic limit, proportional limit, yield strength, tensile strength, fracture strength, ductility, toughness, fatigue ratio or loss coefficient.

7. The method of claim 1, wherein the fabricating the ophthalmic device using the plurality of virtual cross-sections of the three dimensional virtual geometric model to direct an additive manufacturing method further comprises converting the set of physical parameters into control signals to direct one or more laser beams of a stereolithography instrument.

8. The method of claim 1, wherein the additive manufacturing method further comprises three-dimensional printing, stereolithography, microstereolithography, selective laser sintering, direct laser sintering, casting or stamping.

9. The method of claim 1, wherein the fabricating the ophthalmic device using the plurality of virtual cross-sections of the three dimensional virtual geometric model to direct an additive manufacturing method further comprises generation of one or more molds based on the three dimensional virtual geometric model of the ophthalmic device.

10. The method of claim 1, wherein the fabricating the ophthalmic device using the plurality of virtual cross-sections of the three dimensional model to direct an additive manufacturing method further comprises modifying a precursor ophthalmic device.

11. The method of claim 1, wherein the ophthalmic device further comprises a biocompatible polymer, a biodegradable polymer, bio-resistant polymer, biological polymer, photosensitive polymer, a UV curable polymer, cross-linkable polymer, tunable polymer, composite, protein or metal.

12. The method of claim 1, wherein the ophthalmic device further comprises a lens, lens feature, contact lens, hard contact lens, soft contact lens, lens optic, dual optic, haptic, non lens feature, lens positioning ring, accommodating intraocular lens, stent, shunt, punctual plug, retractor, pupil ring, retinal implant, corneal implant, corneal overlay, ophthalmic surgical instrument, sensor or drug eluting device.

13. The method of claim 12, wherein the sensor further comprises a means to obtain glucose concentration, oxygen concentration, electrolyte concentration, chemical analyte concentration, temperature, intraocular pressure, pulse, electrical impedance or eye movement.

14. An ophthalmic device comprising one or more subject specific features generated from receiving one or more images of one or more features of the first eye of the subject; designing a three dimensional virtual geometric model of the ophthalmic device using the one or more images; generating a plurality of virtual cross-sections of the three-dimensional virtual geometric model, wherein the cross-sections are defined by a set of physical parameters derived from the three-dimensional model; and fabricating the one or more subject specific features using the plurality of virtual cross-sections of the three dimensional model to direct an additive manufacturing method.

15. The ophthalmic device of claim 14, wherein the subject specific feature is selected from a group consisting of a lens, lens optic, dual optic, haptic, non lens feature, lens positioning ring, accommodating intra-ophthalmic lens, contact lens, stent, shunt, retractor, retinal implant, corneal implant, ophthalmic surgical instrument, biosensor, and drug elution device.

16. The ophthalmic device of claim 1, wherein the three-dimensional printing method is used to generate a three-dimensional printed mold for manufacturing the subject specific feature of the ophthalmic device.

17. The ophthalmic device of claim 14, wherein the feature is configured to bow when force is applied by the eye inwardly on opposite edges of the feature to generate an accommodating function.

18. The ophthalmic lens device of claim 14, wherein the ophthalmic device is configured to achieve accommodation and/or pseudo-accommodation function in the eye of the subject.

19. The ophthalmic device of claim 1, wherein the feature contains a sensor element.

20. The ophthalmic device of claim 19, wherein the sensor element is configured to measure an analyte selected from the following group consisting of: glucose, oxygen, electrolyte and chemical analyte.

21. The ophthalmic device of claim 19, wherein the sensor element is configured to measure a biophysical parameter selected from the following group consisting of: temperature, intra-ophthalmic pressure, pulse, electrical impedance and eye movement.

22. The ophthalmic lens device of claim 1, wherein the feature contains an element configured to elute a drug.

23. The ophthalmic device of claim 14, wherein the ophthalmic device contains a material selected from the group consisting of: a biocompatible polymer, a UV sensitive reagent, a curing agent, a UV induced cross linker and a chemical catalyst.

24. The ophthalmic device of claim 1, wherein the lens device is configured to be folded about one or more axis(es).

25. The ophthalmic device of claim 1, wherein the feature is configured to change shape when force is applied by the eye on opposite edges of the feature and/or pressure is increased behind the ophthalmic device.

26. The ophthalmic device of claim 1, wherein the lens device is configured to biomimic a natural animal crystalline lens.

27. A system comprising:
a. computer system configured to receive one or more images of features of the first eye of a subject;
b. a means for designing a three dimensional geometric model of an ophthalmic device based on the one or more images;
a means for mathematically slicing the three dimensional geometric model into a plurality of cross sections, wherein the cross-sections are defined by a set of physical parameters derived from the three-dimensional model; and
c. a means for fabricating the ophthalmic device using the plurality of virtual cross-sections of the three dimensional model to direct an additive manufacturing method.
